(19) United States
(12) Patent Application Publication
(10) Pub. No.: US 2023/0348608 A1
(43) Pub. Date: Nov. 2, 2023

(54) Treatment Of Arthropathy Based Upon Stratification Of Osteoarthritis Polygenic Risk Score

(71) Applicant: Regeneron Pharmaceuticals, Inc., Tarrytown, NY (US)

(72) Inventors: Charles Paulding, Tarrytown, NY (US); Nan Lin, Tarrytown, NY (US)

(21) Appl. No.: 18/307,360

(22) Filed: Apr. 26, 2023

Related U.S. Application Data
(60) Provisional application No. 63/335,445, filed on Apr. 27, 2022.

(51) Int. Cl. G16B 20/20; C07K 16/28; A61P 19/02; G16H 50/30

(52) U.S. Cl. CPC C07K 16/2875; A61P 19/02; G16B 20/20; G16H 50/30

(57) ABSTRACT

The present disclosure provides methods of avoiding arthritic side effects of pain treatments, in particular, the side effects of osteoarthritis (OA) pain treatments, through identification of subjects who are likely to suffer the pain treatment related side effects.

### FIELD

The present disclosure is directed to methods of avoiding arthritic side effects of pain treatments, in particular, the side effects of osteoarthritis (OA) pain treatments, by identification of subjects who are likely to suffer the pain treatment related side effects.

### BACKGROUND

Polygenic risk scores (PRSs) combine information from a large number of genetic variants derived from disease association studies to create a single composite quantitative measure for each individual which reflects their genetically-derived disease risk. By aggregating and quantifying the effect of many common variants (usually defined as minor allele frequency ≥1%) in the genome, each individual variant can have a small effect on a person’s genetic risk for a given disease or condition. A PRS is typically constructed as the weighted sum of a collection of genetic variants, usually single nucleotide polymorphisms (SNPs) defined as single base-pair variations from the reference genome. The resulting score is approximately normally distributed in the general population, with higher scores indicating higher risk. With the increasing availability of genetic data in large cohort studies, inclusion of this genetic risk as a covariate in statistical analyses is becoming more widespread. Previously this required specialist knowledge, but as tools and data availability have improved it has become more feasible to calculate scores for use in analyses.

Osteoarthritis is the most common form of arthritis, affecting an estimated 303 million people globally in 2017, and pain is the predominant symptom associated with this disease. OA is a degenerative disease of the synovial joints including the knee, hip, facet joints of the spine, and hand. Risk factors for OA include aging, prior joint injury, obesity, female sex, and genetics. These risk factors are related to the underlying pathogenesis of OA, which is a complex process impacted by altered biomechanics, chronic low-level inflammation, and aging. All of these processes can promote degradation and remodeling of the joint tissues, which ultimately results in failure of the structural integrity of the joint. Although these arthropathy and arthritis do not directly lead to death, they are painful due to the progress of cartilage and bone destruction over time and cause limb dysfunction, which has a great impact on daily life.

There are two general categories of medication for the treatment of pain, each acting via different mechanisms and having differing effects, and both having disadvantages. The first category includes the nonsteroidal anti-inflammatory drugs (NSAIDs) which are used to treat mild pain, but whose therapeutic use is limited by undesirable gastrointestinal effects such as gastric erosion, formation of peptic ulcer or inflammation of the duodenum and of the colon and renal toxicity with prolonged use. The second category includes the opioid analgesics, such as oxycodone, which are used to treat moderate to severe pain but whose therapeutic use is limited because of undesirable effects such as constipation, nausea and vomiting, respiratory depression, mental clouding, renal colic, tolerance to prolonged use and risk of addiction.

Nerve growth factor (NGF) was the first neurotrophin identified, and its role in the development and survival of both peripheral and central neurons has been well characterized. NGF has been shown to be a critical survival and maintenance factor in the development of peripheral sympathetic and embryonic sensory neurons and of basal forebrain cholinergic neurons (Smeyne et al., Nature, 1994, 368, 246-249; and Crowley et al., Cell, 1994, 76, 1001-1011). NGF upregulates expression of neuropeptides in sensory neurons (Lindsay et al., Nature, 1989, 337, 362-364) and its activity is mediated through two different membrane-bound receptors, the TrkA tyrosine kinase receptor and the p75 receptor, which is structurally related to other members of the tumor necrosis factor receptor family (Chao et al., Science, 1986, 232, 518-521).

Fasinumab is a recombinant, fully human, IgG4 anti-NGF monoclonal antibody that binds selectively to NGF without affecting signaling via other neurotrophins, such as neurotrophin 3 and brain-derived neurotrophic factor. Fasinumab and other NGF antagonists have demonstrated the ability to reduce or eliminate pain in subjects suffering from OA-associated pain in clinical trials. However, some patients undergoing NGF antagonist treatment have developed joint damage defined as adjudicated arthropathy (AA) (RPOA1, RPOA2, or DA) or leading to need for joint replacement, such as total joint replacement (TJR), while other treated subjects have displayed no adverse side effects. Accordingly, there is a need to identify a subcategory of patients that would not develop adverse arthropathic side effects and, therefore, would derive the greatest benefits from NGF antagonist treatment.

### SUMMARY

The present disclosure provides methods of treating a subject with OA or at risk of developing OA, the methods comprising: administering a Nerve Growth Factor (NGF) antagonist and/or a therapeutic agent that treats OA to the subject when the subject’s OA-PRS is less than a threshold OA-PRS, wherein the OA-PRS comprises a weighted aggregate of a plurality of genetic variants associated with TJR or AA in subjects treated with an NGF antagonist.

The present disclosure also provides methods of treating a subject having OA, or at risk of developing OA, the methods comprising: administering an analgesic in a standard amount or greater and/or administering a therapeutic agent that treats OA to the subject when the subject’s OA-PRS is greater than or equal to a threshold OA-PRS, wherein the OA-PRS comprises a weighted aggregate of a plurality of genetic variants associated with TJR or AA in subjects treated with an NGF antagonist.

The present disclosure also provides methods of determining whether a subject with OA should be administered an NGF antagonist, the methods comprising: determining or having determined the subject’s OA-PRS, wherein the OA-PRS comprises a weighted aggregate of a plurality of genetic variants associated with TJR or AA in subjects treated with an NGF antagonist; wherein when the subject’s OA-PRS is greater than or equal to a threshold OA-PRS, the subject should be administered an analgesic in a standard amount or greater and/or administered a therapeutic agent that treats OA; or when the subject’s OA-PRS is less than the threshold OA-PRS, the subject should be administered an NGF antagonist and/or a therapeutic agent that treats OA.

The present disclosure also provides methods of assessing a risk of developing joint damage in a subject being treated with an NGF antagonist, the methods comprising: determining or having determined the subject’s OA-PRS, wherein the OA-PRS comprises a weighted aggregate of a plurality of genetic variants associated with TJR or AA in subjects treated with an NGF antagonist; wherein when the subject’s OA-PRS is greater than or equal to a threshold OA-PRS, the subject has an increased risk of developing joint damage; and when the subject’s OA-PRS is less than the threshold OA-PRS, the subject has a decreased risk of developing joint damage.

### BRIEF DESCRIPTION OF THE DRAWINGS

The accompanying figures, which are incorporated in and constitute a part of this specification, illustrate several features of the present disclosure.

FIG. 1 shows knee/hip OA PRS versus Major Safety Endpoints (AA and TJR) in fasinumab treated patients.

FIG. 2 shows knee/hip OA PRS with cumulative incidence of AA events in patients treated (all doses) with fasinumab (Panel A) and knee/hip OA PRS with cumulative incidence of TJR events in patients treated (all doses) with fasinumab (Panel B).

FIG. 3 shows a number of TJR events is higher in AA cases with high OA PRS risk.

FIG. 4 shows knee/hip OA PRS versus Major Safety Endpoints (AA and TJR) in fasinumab 1 mg Q4W treated patients having 1-2 OA joints.

FIG. 5 shows knee/hip OA PRS with cumulative incidence of TJR events in fasinumab 1 mg Q4W treated patients having 1-2 OA joints.

FIG. 6 shows knee/hip OA PRS with cumulative incidence of TJR events by study in fasinumab 1 mg Q4W treated patients having 1-2 OA joints (Panel A) and knee/hip OA PRS versus TJR events by study in fasinumab 1 mg Q4W treated patients having 1-2 OA joints (Panel B).

FIG. 7 shows knee/hip OA PRS with cumulative incidence of AA events in fasinumab 1 mg Q4W treated patients having 1-2 OA joints.

FIG. 8 shows knee/hip OA PRS with cumulative incidence of AA events in fasinumab 1 mg Q4W treated patients having 1-2 OA joints by study.

FIG. 9 shows knee/hip OA PRS vs AA and TJR in patients under fasinumab 1 mg Q4W treatment in 1611 Year 2 study.

FIG. 10 shows AA rate in OA-1611 year 2 study with low dose (1 mg Q4W) and lower OA joint counts (1-2 joints), placebo, and PRS (Panel A) and TJR rate in OA-1611 year 2 study with low dose (1 mg Q4W) and lower OA joint counts (1-2 joints), placebo, and PRS (Panel B).

FIG. 11 shows AA rate in combined long term studies (OA-1611 and PN-1523) comparing low dose (1 mg Q4W) and lower OA joint counts (1-2 joints), placebo, and PRS.

FIG. 12 shows AA rate in PN-1523 with low dose (1 mg Q4W) and lower OA joint counts (1-2 joints) and PRS.

FIG. 13 shows AA rate in OA-1611 with low dose (1 mg Q4W) and lower OA joint counts (1-2 joints) and PRS.

FIG. 14 shows knee/hip OA PRS vs AA or TJR in placebo treated patients.

FIG. 15 shows knee/hip OA PRS vs AA or TJR in NSAIDs treated patients.

FIG. 16 shows a summary of 37 COJO SNP in EUR.

### DESCRIPTION OF EMBODIMENTS

Genetic factors can play an important role in a risk of developing a disease and potentially influence how individuals respond to drug treatment. Polygenic risk scores (PRSs) combine information from a large number of genetic variants derived from disease association studies to create a single composite quantitative measure for each individual which reflects their genetically-derived disease risk. An individual with a larger number of risk alleles for a particular disease will have a higher PRS than an individual with fewer alleles for the same particular disease. Risk can be evaluated at several thresholds, such as percentiles, standard deviation units of the population distribution, or absolute values. The present disclosure relates generally to the unexpected finding that stratification of subjects by Osteoarthritis PRS (OA-PRS) is useful in the identification of subjects likely to avoid arthropathic side effects of NGF antagonists in the treatment of OA pain.

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning - A Laboratory Manual, 2nd ed., Cold Spring Harbor Press (1989), using commercially available reagents, except where otherwise noted.

The present disclosure relates generally to methods and compositions for treating a subject having a risk of developing osteoarthritis.

The present disclosure also relates to methods of avoiding arthropathic side effects of OA treatments by identifying a subgroup of subjects that will not display side effects upon treatment.

Without being limited by any particular theory, it is believed that the OA-PRS calculated, for example, according to the methods presented herein allow for identification of subjects likely to respond to NGF antagonist while avoiding arthropathic side effects.

In some embodiments, a subject who is treatable by the methods of the present disclosure has had OA within the past 1, 2, 3, 4, 5, 6, 7, 8, 9, 10, 11, 12, 13, 14, 15, 16, 17, or 18 months. The subjects who are treatable by the methods of the present disclosure include subjects that have been hospitalized with OA-related symptoms and subjects that are currently hospitalized.

As used herein, the term “nerve growth factor” and “NGF” refers to nerve growth factor and variants (including, for example, splice variants and protein processing variants) thereof that retain at least part of the activity of NGF. As used herein, NGF includes all mammalian species of native sequence NGF, including human, non-human primate, canine, feline, equine, or bovine.

As used herein, the term “antagonist” means either that a given compound is capable of inhibiting the activity of the respective protein or other substance in the cell at least to a certain amount. This can be achieved by a direct interaction of the compound with the given protein or substance (“direct inhibition”) or by an interaction of the compound with other proteins or other substances in or outside the cell which leads to an at least partial inhibition of the activity of the protein or substance (“indirect inhibition”). Inhibition of protein activity can also be achieved through suppressing the expression of a target protein. Techniques of inhibiting protein expression include, but not limited to, antisense inhibition, siRNA-mediated inhibition, miRNA mediated inhibition, ribozyme-mediated inhibition, DNA-directed RNA interference (DdRNAi), RNA-directed DNA methylation, transcription activator-like effector nucleases (TALEN)-mediated inhibition, zinc finger nuclease-mediated inhibition, aptamer-mediated inhibition, and CRISPR-mediated inhibition.

An “NGF antagonist” refers to any molecule that blocks, suppresses, or reduces (including significantly) NGF biological activity, including downstream pathways mediated by NGF signaling, such as receptor binding and/or elicitation of a cellular response to NGF. The term “antagonist” implies no specific mechanism of biological action whatsoever, and is deemed to expressly include and encompass all possible pharmacological, physiological, and biochemical interactions with NGF whether direct or indirect, or whether interacting with NGF, its receptor, or through another mechanism, and its consequences which can be achieved by a variety of different, and chemically divergent, compositions. Exemplary NGF antagonists include, but are not limited to, an anti-NGF antibody, an antisense molecule directed to an NGF (including an antisense molecule directed to a nucleic acid encoding NGF), an NGF antagonist compound, an NGF structural analog, a dominant-negative mutation of a TrkA receptor that binds an NGF, a TrkA immunoadhesin, an anti-TrkA antibody, an anti-p75 antibody, an anti-sense molecule directed to either or both of the TrkA and/or p75 receptors (including anti-sense molecules directed to a nucleic acid molecule encoding TrkA or p75), and a kinase inhibitor. For purpose of the present disclosure, it should be understood that the term “antagonist” encompass all the previously identified terms, titles, and functional states and characteristics whereby the NGF itself, an NGF biological activity (including but not limited to its ability to mediate any aspect of pain), or the consequences of the biological activity, are substantially nullified, decreased, or neutralized in any meaningful degree. In some embodiments, an NGF antagonist binds (physically interact with) NGF (e.g., an antibody), binds to an NGF receptor (such as trkA receptor or p75 receptor), reduces (impedes and/or blocks) downstream NGF receptor signaling, and/or inhibits (reduces) NGF synthesis, production or release. In some embodiments, an NGF antagonist binds (physically interacts with) NGF (e.g., an antibody), binds to an NGF receptor (such as TrkA receptor or p75 receptor), and/or reduces (impedes and/or blocks) downstream NGF receptor signaling. In other embodiments, an NGF antagonist binds NGF and prevents TrkA receptor dimerization and/or TrkA autophosphorylation. In other embodiments, an NGF antagonist inhibits or reduces NGF synthesis and/or production (release). Examples of types of NGF antagonists are provided herein.

In some embodiments, AA can be rapidly progressive osteoarthritis type 1 (RPOA-1) (e.g., joint space narrowing), rapidly progressive osteoarthritis type 2 (RPOA-2) (e.g., limited/partial joint collapse; bone damage), primary osteonecrosis (e.g., avascular necrosis), subchondral insufficiency fracture (SIF), or destructive arthropathy (DA) (e.g., complete joint collapse). In some embodiments, the AA is RPOA-1. In some embodiments, the AA is RPOA-2. In some embodiments, the AA is primary osteonecrosis. In some embodiments, the AA is SIF. In some embodiments, the AA is DA.

In some embodiments, RPOA-1 can be characterized as a rapid loss of joint space width from baseline without evidence of bone fragmentation or destruction. If rapid loss of JSW from baseline is observed by X-ray, an MRI can be obtained and substantial focal or diffuse loss of hyaline/articular cartilage from baseline consistent with RPOA 1 can be observed. A rapid change in joint space width from baseline is defined as: a) knee joints: if JSW is ≥ 2 mm at baseline, a decrease of ≥ 2 mm or 50% from baseline JSW at any point during the study (whichever is greater); and if JSW is < 2 mm at baseline or where accurate JSW measurement is not possible, a change in JSW to 0 mm; b) hip joints: if JSW is ≥ 1.5 mm at baseline, a decrease of > 1.5 mm from baseline; and if JSW is < 1.5 mm at baseline or where accurate JSW measurement is not possible, a change in JSW to 0 mm. If a prior image of the same joint is not available for comparison, by definition, RPOA Type 1 cannot be determined.

In some embodiments, RPOA-2 can be characterized as abnormal bone fragmentation or destruction over a short period of time, including limited collapse of at least one articular surface, and are observed principally by MRI but may be detected by X-rays.

In some embodiments, primary osteonecrosis can be characterized as focal circumscribed or extended region of mottled radiolucency and sclerosis (infarcted bone) which is confirmed by MRI. No evidence of subchondral collapse or bone fragmentation preceding or concurrent with the diagnosis of primary ON.

In some embodiments, SIF can be characterized as subchondral radiolucency, which may have a sclerotic linear component and articular surface flattening and is confirmed by MRI. Does not include significant collapse or fragmentation.

In some embodiments, DA can be characterized as abnormal bone fragmentation, destruction or fracture over a short period of time, including near-total collapse of an articular surface, and often associated with subluxation or malalignment, all of which are features inconsistent with radiographic findings typically observed in conventional advanced OA, and are readily observed by x-rays.

In some embodiments, development of AA can be monitored by X-ray of affected joint with additional imaging as required. In addition, use of sensitive definitions and prospective imaging can be used. Further, frequent use of MRI at baseline and for adjudication can be used since it is superior to conventional radiography at detecting joint pathology.

In some embodiments, when a subject has DA, particular parameters of treatment can be tailored. For example, a) treatment can be restricted to patient populations that are refractory to or unable to tolerate acetaminophen, NSAIDs, and opioids; b) patients with co-morbidities that may result in an increased risk for destructive arthropathy can be excluded; c) fasinumab exposure can be limited by using lower doses; d) concomitant use of NSAIDs can be restricted; and e) can include robust radiologic monitoring by: i) excluding patients with a history of RPOA, ON, SIF or other joint arthropathies that would place patients at risk of joint destruction; ii) schedule radiographic surveillance; iii) timely evaluation (clinical assessment/ X-rays/ MRI) of any reports of worsening pain in any joint; and iv) consider RPOA/SIF/ON as AESI and study drug to be discontinued.

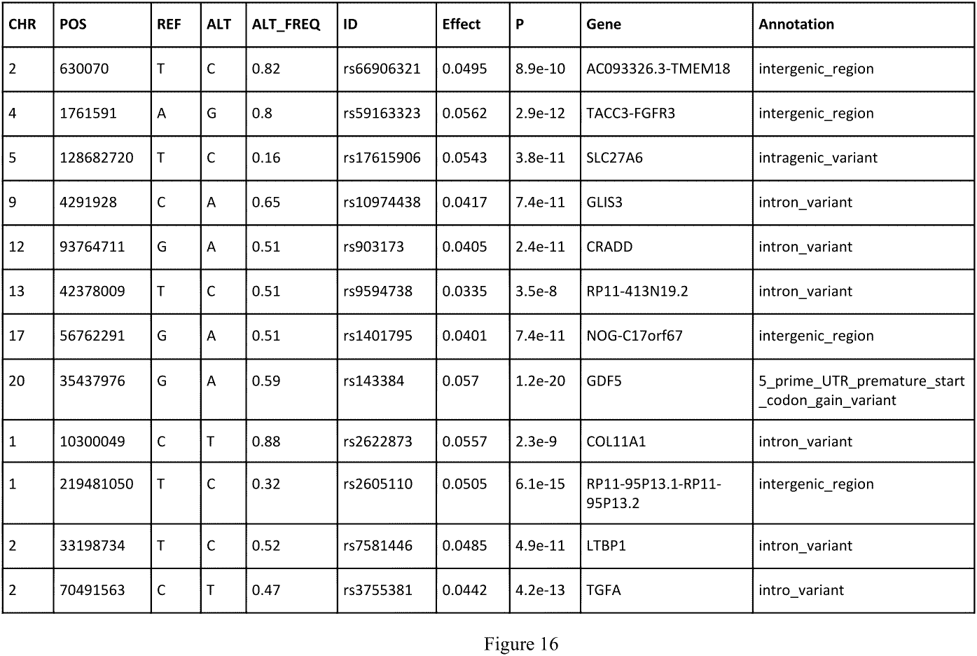

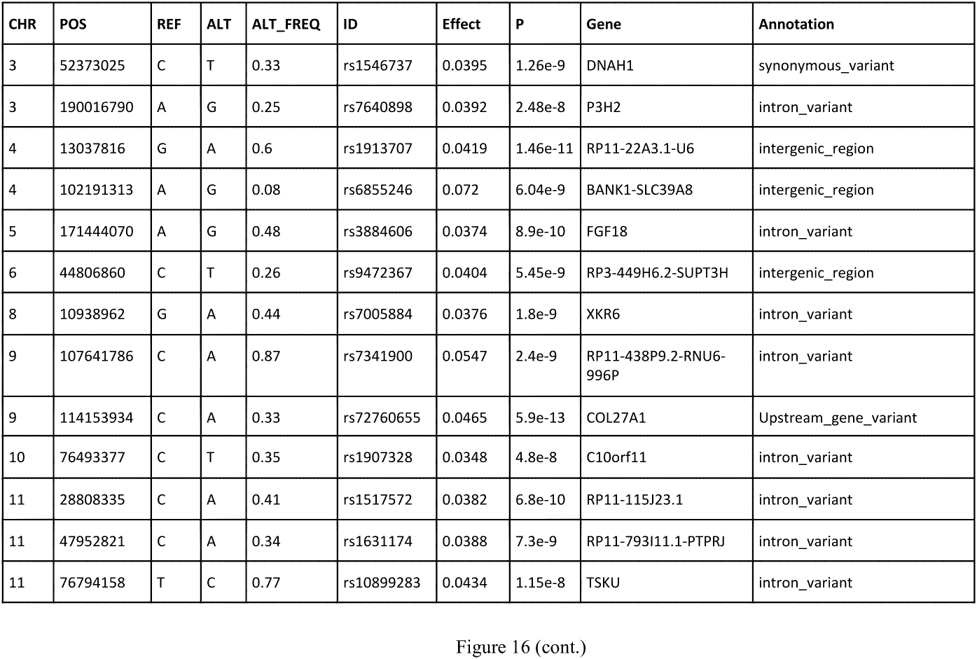

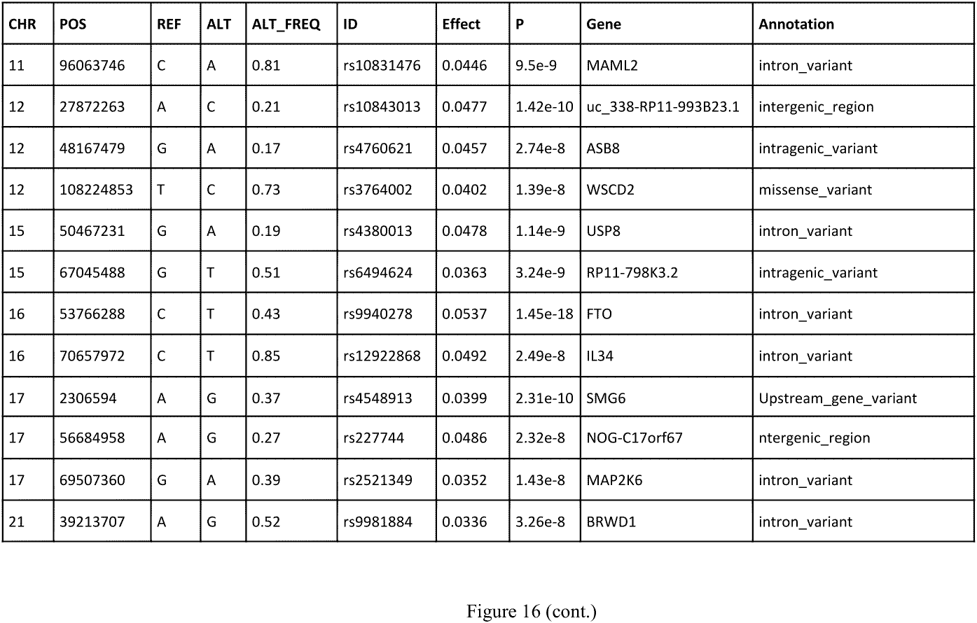

In some embodiments, the subject may be selected on the basis of an OA-PRS, wherein the OA-PRS comprises an aggregate or weighted aggregate of a plurality of genetic variants associated with TJR and/or AA, and is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000 genetic variants, at least about 5,000 genetic variants, at least about 25,000 genetic variants, at least about 50,000 genetic variants, at least about 100,000 genetic variants, at least about 250,000 genetic variants, at least about 500,000 genetic variants, at least about 750,000 genetic variants, at least about 1,000,000 genetic variants, at least about 5,000,000 genetic variants, or at least about 10,000,000 genetic variants. If the subject has an OA-PRS that is less than a threshold OA-PRS, the subject should be administered an NGF antagonist. Alternatively, if the subject has an OA-PRS greater than or equal to a threshold OA-PRS, the subject should be administered an analgesic in a standard amount or greater, and should not be administered an NGF antagonist. In some embodiments, the aggregate of a plurality of genetic variants associated with TJR and/or AA is a weighted aggregate of a plurality of genetic variants associated with TJR and/or AA. In some embodiments, the genetic variants are chosen from any one or more of the variants: rs66906321, rs59163323, rs17615906, rs10974438, rs903173, rs9594738, rs1401795, rs143384, rs2622873, rs2605110, rs7581446, rs3755381, rs1546737, rs7640898, rs1913707, rs6855246, rs3884606, rs9472356, rs7005884, rs7341900, rs72760655, rs1907328, rs1517572, rs1631174, rs10899283, rs10831476, rs10843013, rs4760621, rs3764002, rs4380013, rs6494624, rs9940278, rs12922868, rs4548913, rs227744, rs2521349, and rs9981884 (see, FIG. 16).

Risk assessments using large numbers of genetic variants offers the advantage of increased predictive power. In some embodiments, one or more of the genetic variants is a single nucleotide polymorphism (SNP). In some embodiments, one or more of the genetic variants is an insertion. In some embodiments, one or more of the genetic variants is a deletion. In some embodiments, one or more of the genetic variants is a structural variant. In some embodiments, one or more of the genetic variants is a copy-number variation.

In some embodiments, the present disclosure provides methods of determining an OA-PRS for a subject, the methods comprising identifying whether at least about 2 genetic variants, at least about 5 genetic variants, at least about 10 genetic variants, at least about 15 genetic variants, at least about 20 genetic variants, at least about 30 genetic variants, at least about 40 genetic variants, at least about 50 genetic variants, at least about 60 genetic variants, at least about 70 genetic variants, at least about 100 genetic variants, at leastabout 200 genetic variants, at least about 500 genetic variants, at least about 1000 geneticvariants, at least about 5,000 genetic variants, at least about 25,000 genetic variants, at least about 50,000 genetic variants, at least about 100,000 genetic variants, at least about 250,000 genetic variants, at least about 500,000 genetic variants, at least about 750,000 genetic variants, at least about 1,000,000 genetic variants, at least about 5,000,000 genetic variants, or at least about 10,000,000 genetic variants associated with a risk of developing severe OA requiring joint replacement or developing AA are present in a biological sample from the subject. The presence of a risk allele increases the subject’s OA-PRS.

In some embodiments, the disclosure provides methods of determining an OA-PRS for a subject comprising identifying whether one or more genetic variants associated with a risk of developing severe OA requiring joint replacement or developing AA are present in a biological sample from the subject and calculating an OA-PRS for the subject based on the identified genetic variants, wherein the OA-PRS is calculated by aggregating, such as by summing, the risk score (or weighted risk score) associated with each identified genetic variant. The number of identified genetic variants can be at least about 2 genetic variants, at least about 5 genetic variants, at least about 10 genetic variants, at least about 15 genetic variants, at least about 20 genetic variants, at least about 30 genetic variants, at least about 40 genetic variants, at least about 50 genetic variants, at least about 95 genetic variants, at least about 100 genetic variants, at least about 200 genetic variants, at least about 500 genetic variants, at least about 1000 genetic variants, at least about 5,000 genetic variants, at least about 25,000 genetic variants, at least about 50,000 genetic variants, at least about 100,000 genetic variants, at least about 250,000 genetic variants, at least about 500,000 genetic variants, at least about 750,000 genetic variants, at least about 1,000,000 genetic variants, at least about 5,000,000 genetic variants, or at least about 10,000,000 genetic variants associated with a risk of developing severe OA requiring joint replacement or developing AA. In some embodiments, the disclosure provides methods of determining an OA-PRS for a subject comprising identifying whether the genetic variants associated with a risk of developing severe OA requiring joint replacement or developing AA are present in a biological sample from the subject, wherein the identification process comprises measuring the presence of the at least about 2 genetic variants, at least about 5 genetic variants, at least about 10 genetic variants, at least about 15 genetic variants, at least about 20 genetic variants, at least about 30 genetic variants, at least about 40 genetic variants, at least about 50 genetic variants, at least about 95 genetic variants, at least about 100 genetic variants, at least about 200 genetic variants, at least about 500 genetic variants, at least about 1000 genetic variants, at least about 5,000 genetic variants, at least about 25,000 genetic variants, at least about 50,000 genetic variants, at least about 100,000 genetic variants, at least about 250,000 genetic variants, at least about 500,000 genetic variants, at least about 750,000 genetic variants, at least about 1,000,000 genetic variants, at least about 5,000,000 genetic variants, or at least about 10,000,000 genetic variants.

As an exemplary method, an OA-PRS can be determined from, for example, data obtained from a GWAS of disease risk. For example, in a representative hypothetical GWAS, a GWAS may have identified four genetic variants associated with a disease. Each of the genetic variants may be associated with one or more genes. A value, such as an Odds Ratio, can be calculated for each individual genetic variant. A particular subject’s OA-PRS can be determined by multiplying the log value of the individual Odds Ratio for each variant by the Number Effect Alleles (which is the number of copies of the genetic variant in the genome; i.e., either 0, 1, or 2), and then summing the resultant values. This type of determination can be described by the following Table 1.

TABLE 1

| Gene | Variant rsID | Effect Allele | Odds Ratio (OR) | Number Effect Alleles | Log(OR) × Number Effect Alleles |
|---|---|---|---|---|---|
| A | rs000001 | T | 2.14 | 1 | 0.761 |
| B | rs000002 | A | 1.85 | 0 | 0.000 |
| C | rs000003 | A | 1.36 | 0 | 0.000 |
| ... | ... | ... | ... | ... | ... |
| D | rs000004 | C | 1.28 | 1 | 0.247 |
| Total Score | | | | | 10.910 |

Thus, the subject’s OA-PRS is the sum of the individual values in the last column of the Table taking into consideration any number of genetic variants associated with the particular disease, phenotype, biomarker, laboratory measure, or clinical endpoint. This simplified methodology for determining a subject’s OA-PRS is for exemplary purposes only and shall not be construed to be limiting in any manner. The OA-PRS in the above table is a weighted score because each genetic variant may carry a different weight depending on the particular Odds Ratio and the Number Effect Alleles value.

In some embodiments, the disclosure provides methods of assigning a TJR and/or AA risk group to a subject comprising identifying whether the genetic variants are present in a biological sample from the subject, calculating an OA-PRS for the subject based on the identified genetic variants, and assigning the subject to a risk group based on the OA-PRS. The threshold PRSs can be determined by a hierarchy. In some embodiments, the hierarchy can be by percentiles. By way of a non-limiting example, the OA-PRS may be divided into quintiles, e.g., a top quintile, a top-intermediate quintile, an intermediate quintile, an intermediate-bottom quintile, and a bottom quintile, wherein the top quintile of OA-PRSs correspond the highest genetic risk group and the bottom quintile of OA-PRSs correspond to the lowest genetic risk group. The number of identified genetic variants can be at least about 2 genetic variants, at least about 5 genetic variants, at least about 10 genetic variants, at least about 15 genetic variants, at least about 20 genetic variants, at least about 30 genetic variants, at least about 40 genetic variants, at least about 50 genetic variants, at least about 95 genetic variants, at least about 100 genetic variants, at least about 200 genetic variants, at least about 500 genetic variants, at least about 1000 genetic variants, at least about 5,000 genetic variants, at least about 25,000 genetic variants, at least about 50,000 genetic variants, at least about 100,000 genetic variants, at least about 250,000 genetic variants, at least about 500,000 genetic variants, at least about 750,000 genetic variants, at least about 1,000,000 genetic variants, at least about 5,000,000 genetic variants, or at least about 10,000,000 genetic variants associated with joint replacement or AA.

In some embodiments, the disclosure provides methods for selecting subjects or candidates for administration of NGF antagonist comprising identifying whether at least about 2 genetic variants, at least about 5 genetic variants, at least about 10 genetic variants, at least about 15 genetic variants, at least about 20 genetic variants, at least about 30 genetic variants, at least about 40 genetic variants, at least about 50 genetic variants, at least about 95 genetic variants, at least about 100 genetic variants, at least about 200 genetic variants, at least about 500 genetic variants, at least about 1000 genetic variants, at least about 5,000 genetic variants, at least about 25,000 genetic variants, at least about 50,000 genetic variants, at least about 100,000 genetic variants, at least about 250,000 genetic variants, at least about 500,000 genetic variants, at least about 750,000 genetic variants, at least about 1,000,000 genetic variants, at least about 5,000,000 genetic variants, or at least about 10,000,000 genetic variants are present in a biological sample from the subject or candidate; calculating an OA-PRS for the subject or candidate based on the identified genetic variants; and selecting the subject or candidate for administration of NGF antagonist. In some embodiments, when the subject’s OA-PRS is less a threshold OA-PRS, the subject is identified as a candidate for administration of an NGF antagonist.

In some embodiments, the disclosure provides methods for selecting a population of subjects or candidates for administration of an NGF antagonist, comprising identifying whether at least about 2 genetic variants, at least about 5 genetic variants, at least about 10 genetic variants, at least about 15 genetic variants, at least about 20 genetic variants, at least about 30 genetic variants, at least about 40 genetic variants, at least about 50 genetic variants, at least about 95 genetic variants, at least about 100 genetic variants, at least about 200 genetic variants, at least about 500 genetic variants, at least about 1000 genetic variants, at least about 5,000 genetic variants, at least about 25,000 genetic variants, at least about 50,000 genetic variants, at least about 100,000 genetic variants, at least about 250,000 genetic variants, at least about 500,000 genetic variants, at least about 750,000 genetic variants, at least about 1,000,000 genetic variants, at least about 5,000,000 genetic variants, or at least about 10,000,000 genetic variants associated with joint replacement or AA are present in a biological sample from each subject or candidate of the population of subjects or candidates; calculating an OA-PRS for each subject or candidate based on the identified genetic variants; and selecting the subjects or candidates for administration of an NGF antagonist. In some embodiments, when the subject’s OA-PRS is less a threshold OA-PRS, the subject is identified as a candidate for administration of an NGF antagonist.

In some embodiments, the number of identified genetic variants is at least 4 genetic variants associated with joint replacement or AA. In some embodiments, the number of identified genetic variants is at least 5 genetic variants associated with joint replacement or AA. In some embodiments, the number of identified genetic variants is at least 10 genetic variants associated with joint replacement or AA. In some embodiments, the number of identified genetic variants is at least 20 genetic variants associated with joint replacement or AA. In some embodiments, the number of identified genetic variants is at least 30 genetic variants associated with joint replacement or AA. In some embodiments, the number of identified genetic variants is at least 40 genetic variants associated with joint replacement or AA. In some embodiments, the number of identified genetic variants is at least 50 genetic variants associated with joint replacement or AA. In some embodiments, the number of identified genetic variants is at least 70 genetic variants associated with joint replacement or AA. In some embodiments, the number of identified genetic variants is at least 100 genetic variants associated with joint replacement or AA. In some embodiments, the number of identified genetic variants is at least 500 genetic variants associated with joint replacement or AA. In some embodiments, the number of identified genetic variants is at least 1,000 genetic variants associated with joint replacement or AA. In some embodiments, the number of identified genetic variants is at least 5,000 genetic variants associated with joint replacement or AA. In some embodiments, the number of identified genetic variants is at least 25,000 genetic variants associated with joint replacement or AA. In some embodiments, the number of identified genetic variants is at least 50,000 genetic variants associated with joint replacement or AA. In some embodiments, the number of identified genetic variants is at least 100,000 genetic variants associated with joint replacement or AA. In some embodiments, the number of identified genetic variants is at least 250,000 genetic variants associated with joint replacement or AA. In some embodiments, the number of identified genetic variants is at least 500,000 genetic variants associated with joint replacement or AA. In some embodiments, the number of identified genetic variants is at least 750,000 genetic variants associated with joint replacement or AA. In some embodiments, the number of identified genetic variants is at least 1,000,000 genetic variants associated with joint replacement or AA. In some embodiments, the number of identified genetic variants is at least 5,000,000 genetic variants associated with joint replacement or AA. In some embodiments, the number of identified genetic variants is at least 10,000,000 genetic variants associated with joint replacement or AA.

In some embodiments, risk assessments comprise the highest weighted OA-PRS scores, including, but not limited to the top 50%, 55%, 60%, 70%, 80%, 90%, or 95% of OA-PRS scores from a subject population. In some embodiments of the disclosure, the threshold PRS is a value within the top 50%, 55%, 60%, 70%, 80%, 90%, or 95% percentile of the PRS value. In some embodiments of the disclosure, the threshold PRS is a value within the top 50% percentile of the PRS value. In some embodiments of the disclosure, the threshold PRS is a value within the top 55% percentile of the PRS value. In some embodiments of the disclosure, the threshold PRS is a value within the top 60% percentile of the PRS value. In some embodiments of the disclosure, the threshold PRS is a value within the top 65% percentile of the PRS value. In some embodiments of the disclosure, the threshold PRS is a value within the top 70% percentile of the PRS value. In some embodiments of the disclosure, the threshold PRS is a value within the top 75% percentile of the PRS value. In some embodiments of the disclosure, the threshold PRS is a value within the top 80% percentile of the PRS value. In some embodiments of the disclosure, the threshold PRS is a value within the top 85% percentile of the PRS value. In some embodiments of the disclosure, the threshold PRS is a value within the top 90% percentile of the PRS value. In some embodiments of the disclosure, the threshold PRS is a value within the top 95% percentile of the PRS value.

In some embodiments, the identified genetic variants comprise the highest risk genetic variants or genetic variants with a weighted risk score in the top 10%, top 20%, top 30%, top 40%, top 50%, top 60%, top 70%, or top 75%. In some embodiments, the identified genetic variants comprise the highest risk genetic variants or genetic variants with a weighted risk score in the top 10%. In some embodiments, the identified genetic variants comprise the highest risk genetic variants or genetic variants with a weighted risk score in the top 20%. In some embodiments, the identified genetic variants comprise the highest risk genetic variants or genetic variants with a weighted risk score in the top 30%. In some embodiments, the identified genetic variants comprise the highest risk genetic variants or genetic variants with a weighted risk score in the top 40%. In some embodiments, the identified genetic variants comprise the highest risk genetic variants or genetic variants with a weighted risk score in the top 50%. In some embodiments, the identified genetic variants comprise the highest risk genetic variants or genetic variants with a weighted risk score in the top 60%. In some embodiments, the identified genetic variants comprise the highest risk genetic variants or genetic variants with a weighted risk score in the top 70%. In some embodiments, the identified genetic variants comprise the highest risk genetic variants or genetic variants with a weighted risk score in the top 75%.

In some embodiments, the identified genetic variants comprise the genetic variants having association with total joint replacement in the top 10%, top 20%, top 30%, top 40%, top 50%, top 60%, top 70%, or top 75% of a p-value range. In some embodiments, the identified genetic variants comprise the genetic variants having association with total joint replacement in the top 10% of a p-value range. In some embodiments, the identified genetic variants comprise the genetic variants having association with total joint replacement in the top 20% of a p-value range. In some embodiments, the identified genetic variants comprise the genetic variants having association with total joint replacement in the top 30% of a p-value range. In some embodiments, the identified genetic variants comprise the genetic variants having association with total joint replacement in the top 40% of a p-value range. In some embodiments, the identified genetic variants comprise the genetic variants having association with total joint replacement in the top 50% of a p-value range.. In some embodiments, the identified genetic variants comprise the genetic variants having association with total joint replacement in the top 60% of a p-value range.. In some embodiments, the identified genetic variants comprise the genetic variants having association with total joint replacement in the top 70% of a p-value range.. In some embodiments, the identified genetic variants comprise the genetic variants having association with total joint replacement in the top 75% of a p-value range.

In some embodiments, each of the identified genetic variants comprise genetic variants having association with total joint replacement with a p-value of not larger than about 10⁻¹, about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, or about 10⁻¹⁵. In some embodiments, the identified genetic variants comprise the genetic variants having association with the total joint replacement with p-value of less than 5 × 10⁻⁸.

In some embodiments, the identified genetic variants comprise genetic variants having association with total joint replacement in high-risk subjects as compared to the rest of the reference population with an odds ratio (OR) of about 1.0 or greater, about 1.5 or greater, about 1.75 or greater, about 2.0 or greater, about 2.25 or greater, or about 2.75 or greater for the top half (up to 50%) of the distribution; about 1.5 or greater, about 1.75 or greater, about 2.0 or greater, about 2.25 or greater, about 2.5 or greater, or about 2.75 or greater of the top quarter (up to 55%) of the distribution; about 1.0 or greater, about 1.5 or greater, about 1.75 or greater, about 2.0 or greater, about 2.25 or greater, or about 2.75 or greater for up to 60% of the distribution; about 1.0 or greater, about 1.5 or greater, about 1.75 or greater, about 2.0 or greater, about 2.25 or greater, or about 2.75 or greater for up to 70% of the distribution; about 1.0 or greater, about 1.5 or greater, about 1.75 or greater, about 2.0 or greater, about 2.25 or greater, or about 2.75 or greater for up to 80% of the distribution; about 1.0 or greater, about 1.5 or greater, about 1.75 or greater, about 2.0 or greater, about 2.25 or greater, or about 2.75 or greater for up to 90% of the distribution; or about 1.0 or greater, about 1.5 or greater, about 1.75 or greater, about 2.0 or greater, about 2.25 or greater, or about 2.75 or greater for up to 95% of the distribution. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, or from about 6.5 to about 7.0. In some embodiments, high-risk subjects comprise subjects having OA-PRS scores in the top decile, quintile, or tertile of a reference population.

In some embodiments, the identified genetic variants comprise genetic variants having the highest genetic variant performance in the reference population. In some embodiments, genetic variant performance is calculated with respect to total joint replacement risk based on statistical significance, strength of association, and/or a probability distribution.

In some embodiments, genetic variant scores are calculated using PRS calculation methodologies such as the LDPred method (or variations and/or versions thereof). LDPred is a Bayesian approach to calculate a posterior mean effect for all variants based on a prior (effect size in the prior genome-wide association study) and subsequent shrinkage based on linkage disequilibrium. LDPred creates a PRS using genome-wide variation with weights derived from a set of genome-wide association study (GWAS) summary statistics. See, Vilhjálmsson et al., Am. J. Hum. Genet., 2015, 97, 576-92. In some embodiments, alternate approaches for calculating genetic variant scores may be used, including SBayesR (Lloyd-Jones, LR, world wide web at “biorxiv.org/content/biorxiv/early/2019/01/17/ 522961.full.pdf”), Pruning and Thresholding (P&T) (Purcell, Nature, 2009, 460, 748-752), and conditional and joint analyses (COJO) (Yang et al., Nat. Genet., 2012, 44, 369-375). SBayesR is a Bayesian approach is similar to LDPred but allows for more flexibility in the posterior mean effects. Pruning and Thresholding (P&T) requires that a minimum p-value threshold (p-value associated with the variant from the source data file) and r2 threshold (measure of linkage disequilibrium (LD)) between variants be specified. P&T identifies the variant with the smallest p-value in each region and then “clumps” under that variant all other variants in the region with an r2 value that is larger than the specified r2. In the PRS, the index variant represents all the variants in the clump (only the index variant is included in the PRS with all other variants are excluded). COJO is similar conceptually to P&T but incorporates additional variants in a given LD block into the score if they demonstrate independent contribution to disease risk after conditioning on the index variant.

AA and TJR are two adverse events (AEs) being monitored that are significantly associated with higher than threshold OA PRS score. In some embodiments, AA association with PRS score is prominent after the treatment duration is longer than one year (for example Trial 1611), whereas TJR association with PRS score is significant in all duration trials (PN-1523, OA-1611 and OA-1688).

In some embodiments, genetic variant performance is calculated using the LDPred method, wherein the ρ value is from about 0.0001 to about 0.5. In some embodiments, genetic variant performance is calculated using the LDPred method, wherein the ρ value is about 0.5. In some embodiments, genetic variant performance is calculated using the LDPred method, wherein the ρ value is about 0.1. In some embodiments, genetic variant performance is calculated using the LDPred method, wherein the ρ value is about 0.05. In some embodiments, genetic variant performance is calculated using the LDPred method, wherein the ρ value is about 0.01. In some embodiments, genetic variant performance is calculated using the LDPred method, wherein the ρ value is about 0.005. In some embodiments, genetic variant performance is calculated using the LDpred method, wherein the ρ value is about 0.001. In some embodiments, genetic variant performance is calculated using the LDPred method, wherein the ρ value is about 0.0005. In some embodiments, genetic variant performance is calculated using the LDPred method, wherein the ρ value is about 0.0001.

In some embodiments, the method further comprises an initial step of obtaining a biological sample from the subject.

The biological sample may contain whole cells, live cells and/or cell debris. The biological sample may contain (or be derived from) a bodily fluid. The present disclosure encompasses embodiments wherein the bodily fluid is selected from amniotic fluid, aqueous humor, vitreous humor, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammalian organism, for example by venapuncture, or other collecting or sampling procedures.

The present disclosure also provides methods of treating a subject having OA, or at risk of developing OA, the methods comprising: administering an analgesic and/or a therapeutic agent that treats OA to the subject when the subject’s OA-PRS is greater than or equal to a threshold OA-PRS, wherein the OA-PRS comprises a weighted aggregate of a plurality of genetic variants associated with TJR and/or AA in subjects treated with an NGF antagonist.

The present disclosure also provides methods of treating a subject having OA, or at risk of developing OA, the methods comprising: administering an NGF antagonist and/or a therapeutic agent that treats OA to the subject when the subject’s OA-PRS is less than a threshold OA-PRS, wherein the OA-PRS comprises a weighted aggregate of a plurality of genetic variants associated with TJR and/or AA in subjects treated with an NGF antagonist.

The present disclosure also provides methods of determining whether a subject having OA should be administered an NGF antagonist, the methods comprising: determining or having determined the subject’s OA-PRS, wherein the OA-PRS comprises a weighted aggregate of a plurality of genetic variants associated with TJR and/or AA in subjects treated with an NGF antagonist; wherein when the subject’s OA-PRS is greater than or equal to a threshold OA-PRS, the subject should be administered an analgesic in a standard amount or greater.

The present disclosure also provides methods of determining whether a subject having OA should be administered an NGF antagonist, the methods comprising: determining or having determined the subject’s OA-PRS, wherein the OA-PRS comprises a weighted aggregate of a plurality of genetic variants associated with TJR and/or AA in subjects treated with an NGF antagonist; wherein when the subject’s OA-PRS is greater than or equal to a threshold OA-PRS, the subject should be administered an analgesic in a standard amount or greater and/or a therapeutic agent that treats OA.

The present disclosure also provides methods of determining whether a subject having OA should be administered an NGF antagonist, the methods comprising: determining or having determined the subject’s OA-PRS, wherein the OA-PRS comprises a weighted aggregate of a plurality of genetic variants associated with TJR and/or AA in subjects treated with an NGF antagonist; wherein when the subject’s OA-PRS is less than the threshold OA-PRS, the subject should be administered an NGF antagonist.

The present disclosure also provides methods of determining whether a subject having OA should be administered an NGF antagonist, the methods comprising: determining or having determined the subject’s OA-PRS, wherein the OA-PRS comprises a weighted aggregate of a plurality of genetic variants associated with TJR and/or AA in subjects treated with an NGF antagonist; wherein when the subject’s OA-PRS is less than the threshold OA-PRS, the subject should be administered an NGF antagonist and/or a therapeutic agent that treats OA.

The present disclosure also provides methods of determining whether a subject having OA should be administered an analgesic in a standard amount or greater, the methods comprising: determining or having determined the subject’s OA-PRS, wherein the OA-PRS comprises a weighted aggregate of a plurality of genetic variants associated with TJR and/or AA in subjects treated with an NGF antagonist; wherein when the subject’s OA-PRS is greater than or equal to a threshold OA-PRS, the subject should be administered an analgesic in a standard amount or greater.

The present disclosure also provides methods of determining whether a subject having OA should be administered an analgesic in a standard amount or greater, the methods comprising: determining or having determined the subject’s OA-PRS, wherein the OA-PRS comprises a weighted aggregate of a plurality of genetic variants associated with TJR and/or AA in subjects treated with an NGF antagonist; wherein when the subject’s OA-PRS is greater than or equal to a threshold OA-PRS, the subject should be administered an analgesic in a standard amount or greater and/or a therapeutic agent that treats OA.

The present disclosure also provides methods of determining whether a subject having OA should be administered an analgesic in a standard amount or greater, the methods comprising: determining or having determined the subject’s OA-PRS, wherein the OA-PRS comprises a weighted aggregate of a plurality of genetic variants associated with TJR and/or AA in subjects treated with an NGF antagonist; wherein when the subject’s OA-PRS is less than a threshold OA-PRS, the subject should be administered an NGF antagonist.

The present disclosure also provides methods of determining whether a subject having OA should be administered an analgesic in a standard amount or greater, the methods comprising: determining or having determined the subject’s OA-PRS, wherein the OA-PRS comprises a weighted aggregate of a plurality of genetic variants associated with TJR and/or AA in subjects treated with an NGF antagonist; wherein when the subject’s OA-PRS is less than a threshold OA-PRS, the subject should be administered an NGF antagonist and/or a therapeutic agent that treats OA.

The present disclosure also provides methods of assessing risk of developing joint damage in a subject being treated with an NGF antagonist, the methods comprising: determining or having determined the subject’s OA-PRS, wherein the OA-PRS comprises a weighted aggregate of a plurality of genetic variants associated with TJR and/or AA in subjects treated with an NGF antagonist; wherein when the subject’s OA-PRS is greater than or equal to a threshold OA-PRS, the subject has an increased risk of developing joint damage.

The present disclosure also provides methods of assessing risk of developing joint damage in a subject being treated with an NGF antagonist, the methods comprising: determining or having determined the subject’s OA-PRS, wherein the OA-PRS comprises a weighted aggregate of a plurality of genetic variants associated with TJR and/or AA in subjects treated with an NGF antagonist; wherein when the subject’s OA-PRS is less than the threshold OA-PRS, the subject has a decreased risk of developing joint damage.

Any of the methods described herein can be used to select a population of subjects or candidates for clinical trials, e.g., a clinical trial whose patient population is suitable for treatment by NGF antagonist or other OA or pain (such as chronic pain) treatment regimen. In some embodiments, the selected candidates or subjects are divided into subgroups based on the identified genetic variants for each subject or candidate, and the method is used to determine whether a particular treatment or treatment plan is effective for a subject having a particular genetic variant or a particular group of genetic variants. For example, the methods described herein can be employed to determine susceptibility of a population of subjects to a particular treatment or treatment plan, wherein the population of subjects is selected based on the genetic variants identified in the subjects.

In some embodiments, the method is used to select a population of subjects or candidates for clinical trials, e.g., a clinical trial to determine whether a particular NGF antagonist is suitable treatment for OA. In some embodiments, the desired risk group is a population comprising low risk subjects or candidates. In some embodiments, the selected population of subjects or candidates are responders, i.e., the subjects or candidates are responsive to the treatment or treatment plan.

In some embodiments the subjects are selected based on OA-PRS alone. For example, if a subject or a candidate that has an OA-PRS above a pre-determined threshold, the subject is selected for initiating treatment or a candidate is included in the clinical trial. In some embodiments, the threshold for treatment initiation or clinical trial inclusion is determined in relative terms. For example, in some embodiments, the threshold OA-PRS score is the top 50% within a reference population. In some embodiments, the threshold OA-PRS score is the top 40% within a reference population. In some embodiments, the threshold OA-PRS score is the top 30% within a reference population. In some embodiments, the threshold OA-PRS score is the top 25% within a reference population. In some embodiments, the threshold OA-PRS score is the top 20% within a reference population. In some embodiments, the threshold OA-PRS score is the top 15% within a reference population. In some embodiments, the threshold OA-PRS score is the top 10% (decile) within a reference population. In some embodiments, the threshold OA-PRS score is the top 5% within a reference population.

In some embodiments, the reference population for determination of relative OA-PRS score is at least about 100 subjects. In some embodiments, the reference population for determination of relative OA-PRS score is at least about 200 subjects. In some embodiments, the reference population for determination of relative OA-PRS score is at least about 500 subjects. In some embodiments, the reference population for determination of relative OA-PRS score is at least about 1,000 subjects. In some embodiments, the reference population for determination of relative OA-PRS score is at least about 3,000 subjects. In some embodiments, the reference population for determination of relative OA-PRS score is at least about 5,000 subjects. In some embodiments, the reference population for determination of relative OA-PRS score is at least about 7,500 subjects. In some embodiments, the reference population for determination of relative OA-PRS score is at least about 10,000 subjects. In some embodiments, the reference population for determination of relative OA-PRS score is at least about 12,000 subjects. In some embodiments, the reference population for determination of relative OA-PRS score is at least about 15,000 subjects. In some embodiments, the reference population for determination of relative OA-PRS score is at least about 20,000 subjects. In some embodiments, the reference population for determination of relative OA-PRS score is at least about 30,000 subjects. In some embodiments, the reference population for determination of relative OA-PRS score is at least about 50,000 subjects. In some embodiments, the reference population for determination of relative OA-PRS score is at least about 70,000 subjects. In some embodiments, the reference population for determination of relative OA-PRS score is at least about 100,000 subjects.

In some embodiments, the reference population is enriched for members of an ancestry group. In some embodiments, the ancestry group is self-reported. In some embodiments, the ancestry group is assigned based upon genetic testing for ancestry. In some embodiments, the ancestry group is derived from a principal component analysis of ancestry. In some embodiments the ancestry group is European. In some embodiments the ancestry group is African. In some embodiments the ancestry group is admixed American. In some embodiments the ancestry group is East Asian. In some embodiments the ancestry group is South Asian. In some embodiments the ancestry group is any mixture of any two or more of the European, African, admixed American, East Asian, and South Asian populations.

In some embodiments, the any of the methods described herein further comprises initiating OA treatment to the subject. The treatment may comprise administration of an analgesic, a steroid injection, a therapeutic injections, an antidepressant, physical therapy, a strengthening exercise, radiofrequency nerve ablation, and surgery, or any combination thereof.

Examples of analgesics useful for treating OA include, but are not limited to, nonsteroidal anti-inflammatory drugs (NSAIDs, such as ibuprofen, naproxen, diclofenac, etodolac, meloxicam, oxaprozin, celecoxib, or piroxicam), acetaminophen, glucosamine, chondroitin, and opioids (such as codeine, hydrocodone, oxycodone, or tramadol), or any combination thereof.

Examples of steroid injections useful for treating OA include, but are not limited, to corticosteroids (such as triamcinolone, cortisone, prednisone, and methylprednisolone, or any combination thereof).

Examples of therapeutic injections useful for treating OA include, but are not limited to, a hyaluronic acid injection.

Examples of antidepressants useful for treating OA include, but are not limited to, duloxetine, amitriptyline, desipramine, and nortriptyline, or any combination thereof.

In some embodiments, treatment of OA can include physical therapy, cognitive behavioral therapy, and/or weight loss.

In some embodiments, any of the methods described herein further comprise initiating NGF antagonist treatment to the subject having OA-PRS score that is less than a threshold OA-PRS score.

Suitable NGF antagonists suitable for treatment of OA include, but are not limited to, anti-NGF antibodies, polypeptides, antisense nucleic acid molecules, NGF-targeting siRNA, or a small molecule NGF antagonists.

Anti-NGF antibodies are able to bind to NGF and inhibit NGF biological activity and/or downstream pathway(s) mediated by NGF signaling. Numerous anti-NGF antibodies are described in, for example, PCT Publication Nos. WO 00/073344, WO 02/096458, WO 01/78698, and WO 01/64247, U.S. Application Publication No. US2011/0206682; U.S. Pat. Nos. 5,844,092, 5,877,016, and 6,153,189; Hongo et al., Hybridoma, 2000, 19, 215-227; Cell. Molec. Biol., 1993, 13, 559-568; GenBank Accession Nos. U39608, U39609, L17078, and L17077. In some embodiments, the anti-NGF antibody is ABT-110, fasinumab, tanezumab, or fulranumab. In some embodiments, the anti-NGF antibody is fasinumab.

Suitable NGF antagonist polypeptides include, but not limited to, NGF mimetic peptides, which competitively bind TrkA or P75^{NTR} receptors. Numerous NGF antagonist peptides are described, for example, in LeSauteur et al., J. Biol. Chem., 1995, 270, 6564-6569; and Brahimi et al., Biochim. Biophys. Acta, 2010, 1800, 1018-1026; and Longo et al., J. Neurosci. Res., 1997, 48, 1-17; PCT Publication Nos. WO 97/15593 and WO89/09225; and U.S. Pat. Nos. 6,291,247; and No. 6,017,878. An additional NGF antagonist is medi7352.

Suitable small molecule NGF antagonists are described in, for example in U.S. Publication No. 20010046959. Compounds that inhibit NGF’s binding to p75 are described in PCT Publication No. WO 00/69829. Compounds that inhibit NGF’s binding to TrkA/p75 are described in PCT Publication No. WO 98/17278. Additional examples of NGF antagonists include the compounds described in PCT Publication Nos. WO 02/17914 and WO 02/20479, U.S. Pat. Nos. 5,342,942, 6,127,401, and 6,359,130. Further exemplary NGF antagonists are compounds that are competitive inhibitors of NGF. See U.S. Patent No. 6,291,247. In some embodiments, small molecule NGF antagonist is K252a, ALE-0540, PQC-083, PD-90780, LM11A-31 dihydrochloride, Y1036, or Ro 08-2750.

Initiating a treatment can include devising a treatment plan based on the risk group, which corresponds to the OA-PRS calculated for the subject. In some embodiments, an OA-PRS is predictive of treatment efficacy or of a subject’s response to a therapeutic regimen. Accordingly, the treatment can be determined or adjusted according to the OA-PRS.

In some embodiments, the treatment initiation comprises modifying dosage or regimen of a treatment that a subject with OA already receives based on an OA-PRS calculated for the subject. In some embodiments, the treatment initiation comprises substitution of one therapeutic agent with another based on an OA-PRS. In some embodiments, the treatment initiation comprises substitution of an NGF antagonist with an analgesic based on a subject’s having an OA-PRS that is equal to or exceeds a threshold OA-PRS. In some embodiments, the treatment initiation comprises substitution of fasinumab with an analgesic based on a subject’s having an OA-PRS that is equal to or exceeds a threshold OA-PRS. In some embodiments, the treatment initiation comprises starting a regimen of a therapeutic agent in addition to a therapeutic agent a subject already receives. In some embodiments, the treatment initiation comprises starting administration of a therapeutic regimen to a previously untreated OA subject.

Antibodies are intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments, the FRs of the antibody (or antigen-binding fragment thereof) may be identical to the human germline sequences, or may be naturally or artificially modified. An amino acid consensus sequence may be defined based on a side-by-side analysis of two or more CDRs.

Antibodies include antigen-binding fragments of full antibody molecules. An antigen-binding portion of an antibody or an antigen-binding fragment of an antibody include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Antisense inhibition includes reduction of target nucleic acid levels in the presence of an oligonucleotide complementary to a target nucleic acid compared to target nucleic acid levels in the absence of the oligonucleotide.

An effective amount is an amount sufficient to effect beneficial or desired clinical results including alleviation or reduction in the pain sensation. For purposes of the present disclosure, an effective amount of an NGF antagonist (such as an anti-NGF antibody) includes an amount sufficient to treat, ameliorate, reduce the intensity of or prevent pain (including nociception and the sensation of pain) of any sort, including acute, chronic, inflammatory, neuropathic, or post-surgical pain. In some embodiments, an effective amount of an NGF antagonist is a quantity of the NGF antagonist capable of modulating the sensitivity threshold to external stimuli to a level comparable to that observed in healthy subjects. In other embodiments, this level may not be comparable to that observed in healthy subjects, but is reduced compared to not receiving the combination therapy. As is understood in the art, an effective amount of NGF antagonist may vary, depending on, inter alia, type of pain (and patient history as well as other factors such as the type (and/or dosage) or NGF antagonist used.

In the context of the methods disclosed herein, additional therapeutically active component(s), e.g., any of the agents listed above or derivatives thereof, may be administered just prior to, concurrent with, or shortly after the administration of an NGF antagonist; (for purposes of the present disclosure, such administration regimens are considered the administration of an NGF antagonist “in combination with” an additional therapeutically active component). In some embodiments, an additional therapeutically active component is considered administered “in combination with” an NGF antagonist notwithstanding the fact that the additional therapeutically active component and the NGF antagonist are administered by different routes. The present methods include pharmaceutical compositions and methods of use thereof in which an NGF antagonist is co-formulated with one or more of the additional therapeutically active component(s) as described herein.

As used herein, the terms “treat”, “treatment”, or “treating” refers to administering a therapeutic agent for prophylactic and/or therapeutic purposes.

As used herein, “therapeutic treatment” refers to administering a therapeutic agent to a subject having OA.

As used herein, “prophylactic treatment” and “prophylaxis” refer to administration of a subject who is not currently nor ever has had OA.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in °C or is at ambient temperature, and pressure is at or near atmospheric.

### EXAMPLES

### Example 1: Knee/Hip OA PRS Model Building

A knee/hip OA PRS model was constructed using external data (published in Boer et al., Cell, 2021, 184, 4784-4818). Summary statistics of a knee/hip OA genome-wide association analysis of 89,741 OA patients and 400,604 controls were used for the generation of the OA-PRS. After conducting conditional and joint multiple-SNP analysis of the source GWAS, 37 SNPs were identified with corresponding adjusted effect sizes to predict patients’ OA genetic risk.

### Example 2: Combined Population Knee/Hip OA PRS vs Safety Endpoints in Fasinumab Treated Patients

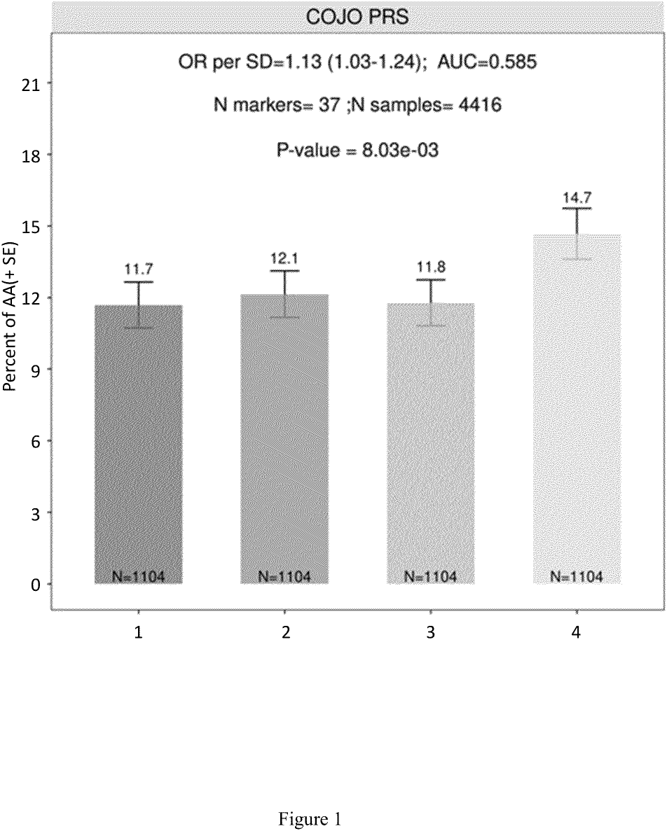

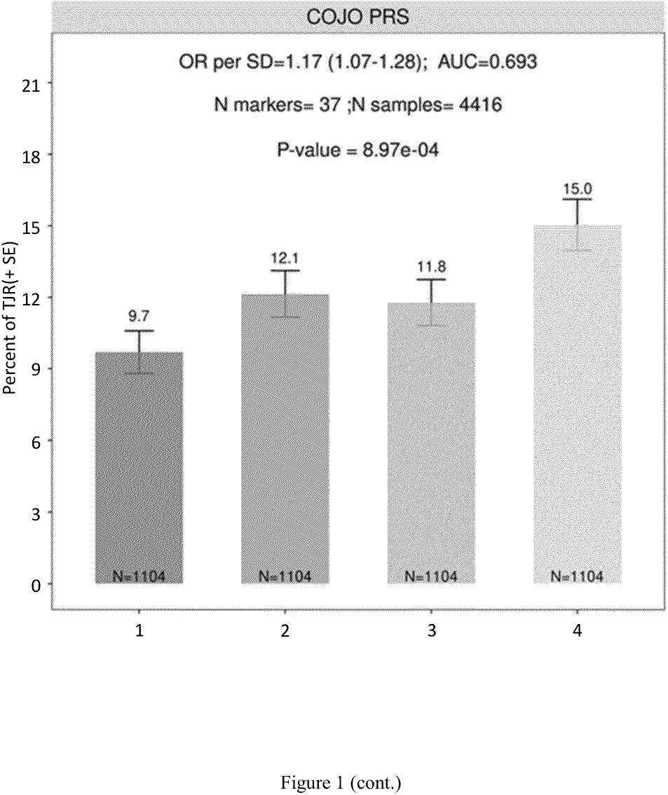

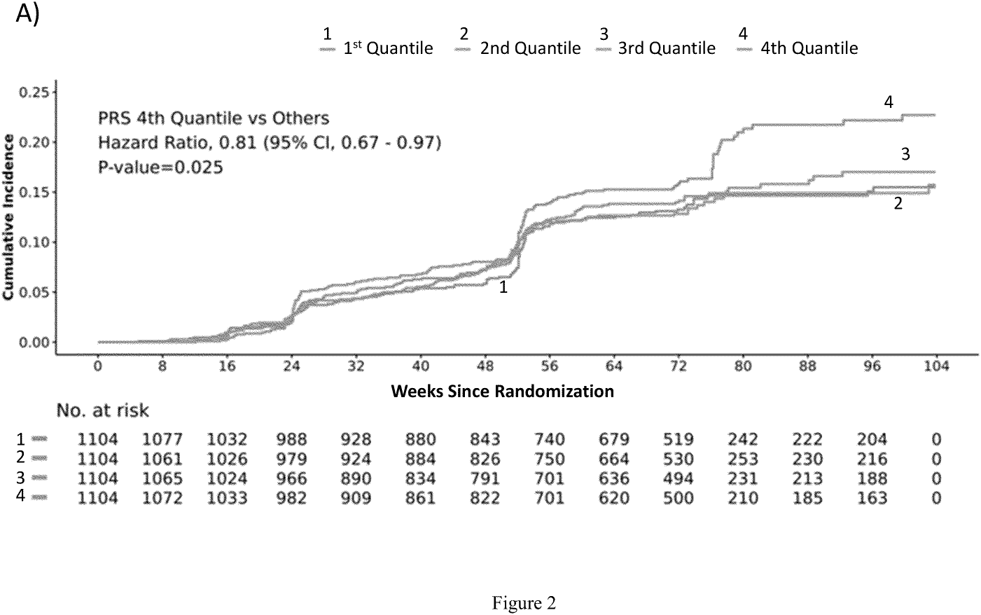

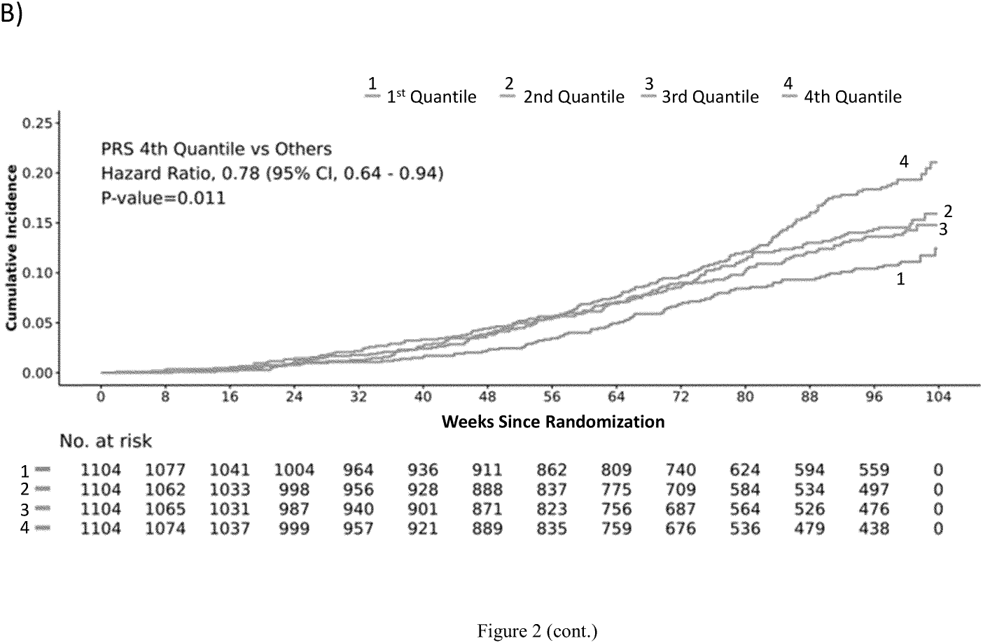

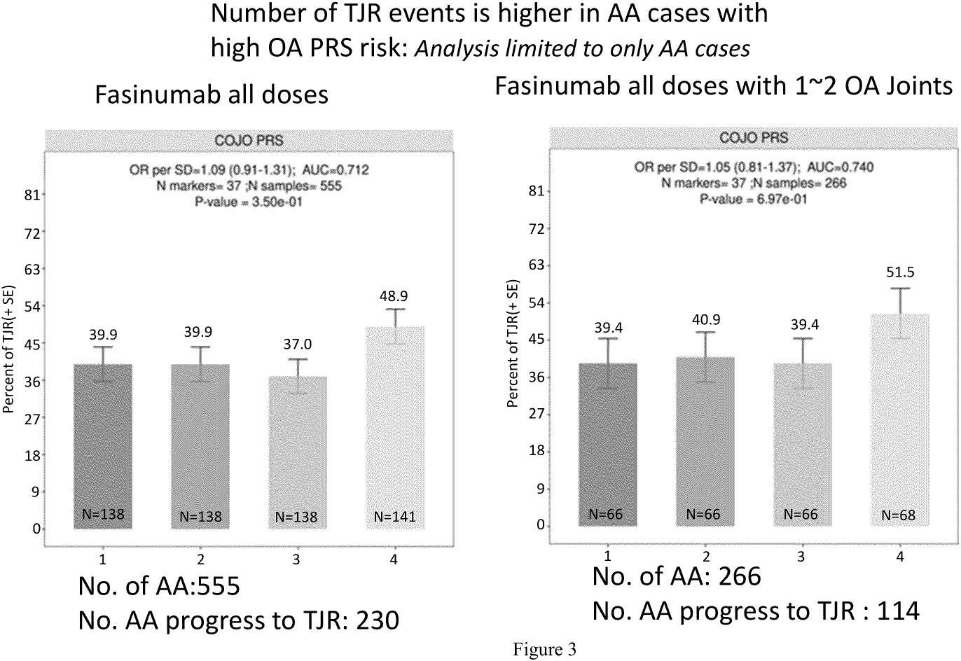

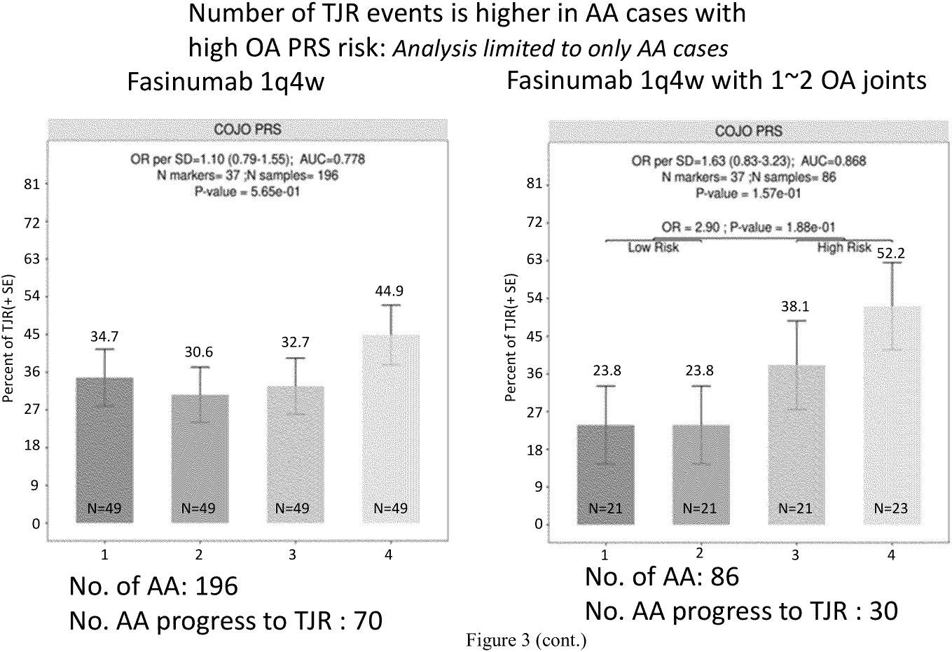

The OA-PRS was robustly associated with the risk of developing AA and TJR (see, FIG. 1). High PRS scores were associated with an increased risk of developing AA and were also associated with an increased risk of having a TJR procedure. OA PRS vs AA and TJR cumulative incidence in fasinumab treated patients (all doses) was also examined (see, FIG. 2, Panel A and Panel B) showing that patients with high OA PRS risk have an increase in AA and TJR events. FIG. 3 shows a number of TJR events is higher in AA cases with high OA PRS risk, indicating that excluding patients with high OA PRS risk could provide additional safety for patients with AA events, keeping the risk of TJR progression lower.

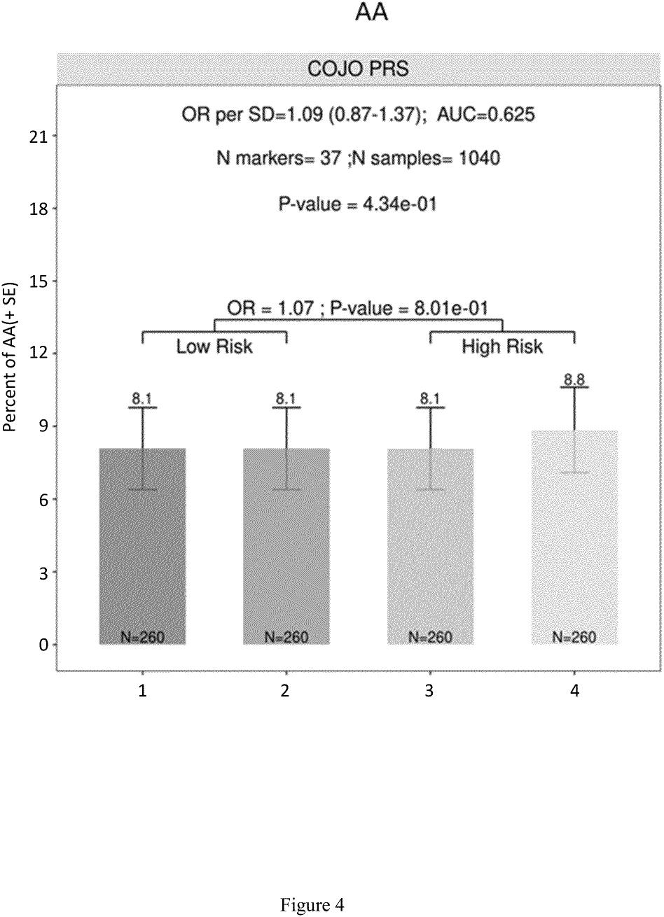

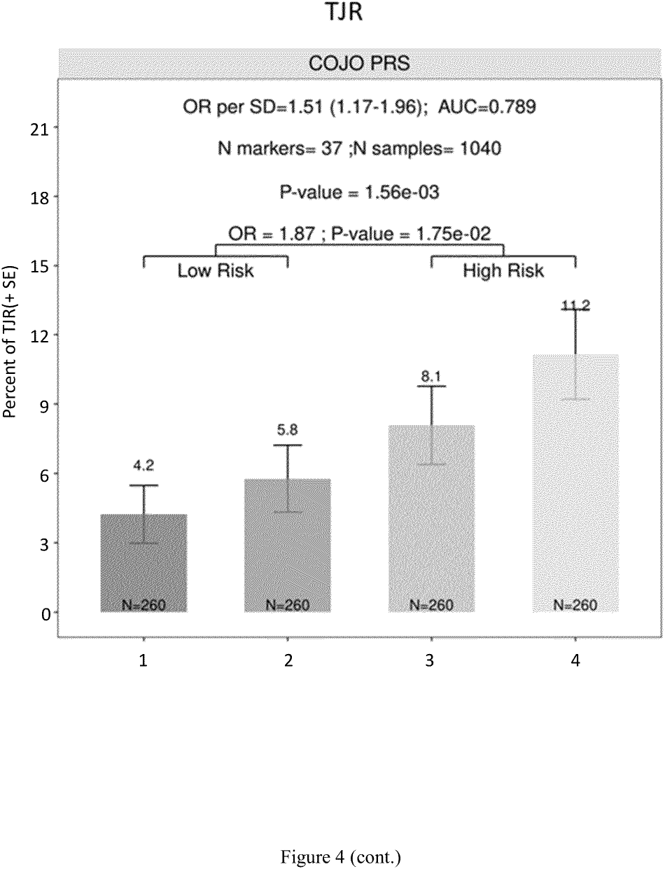

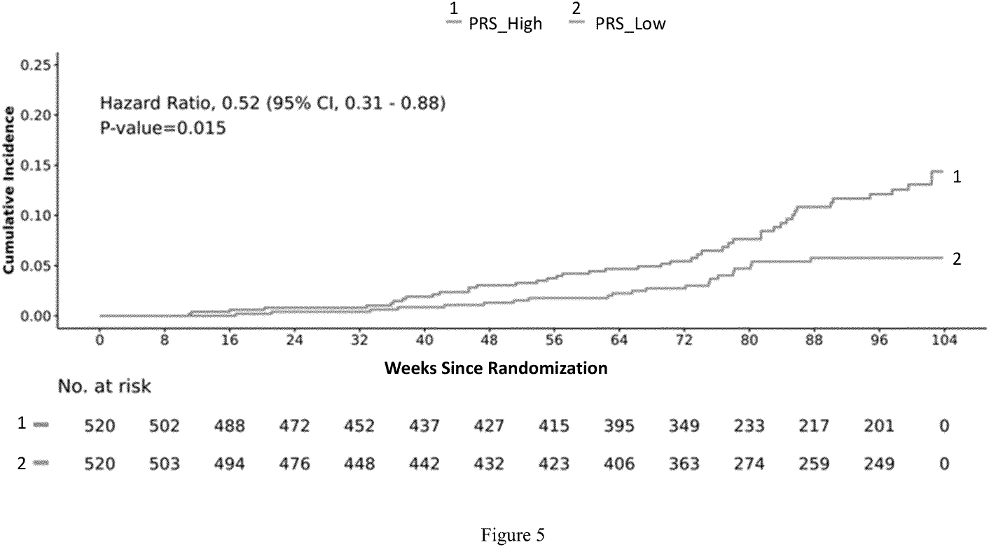

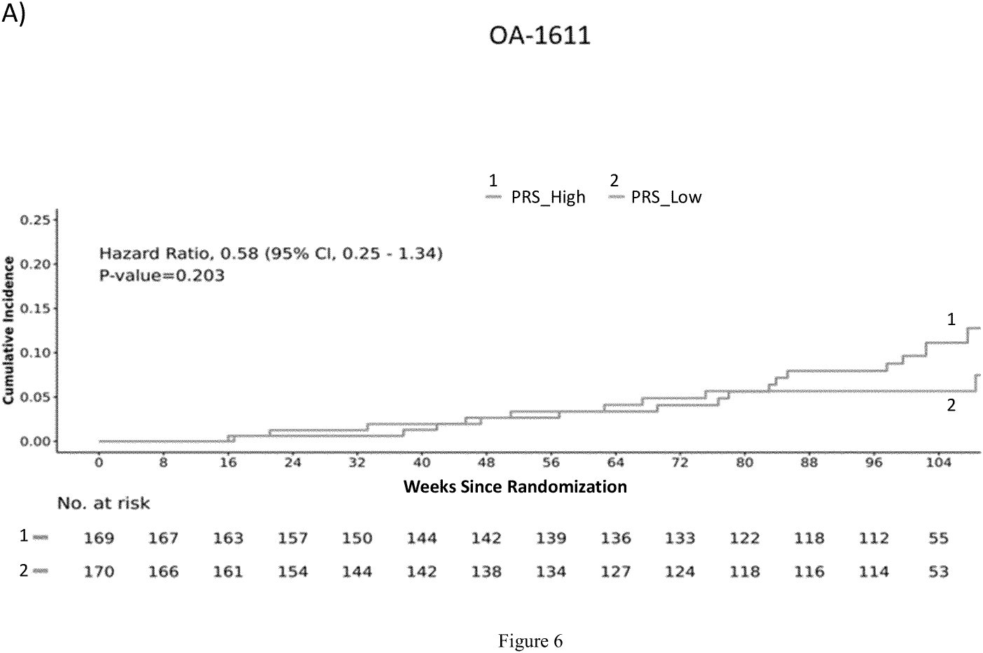

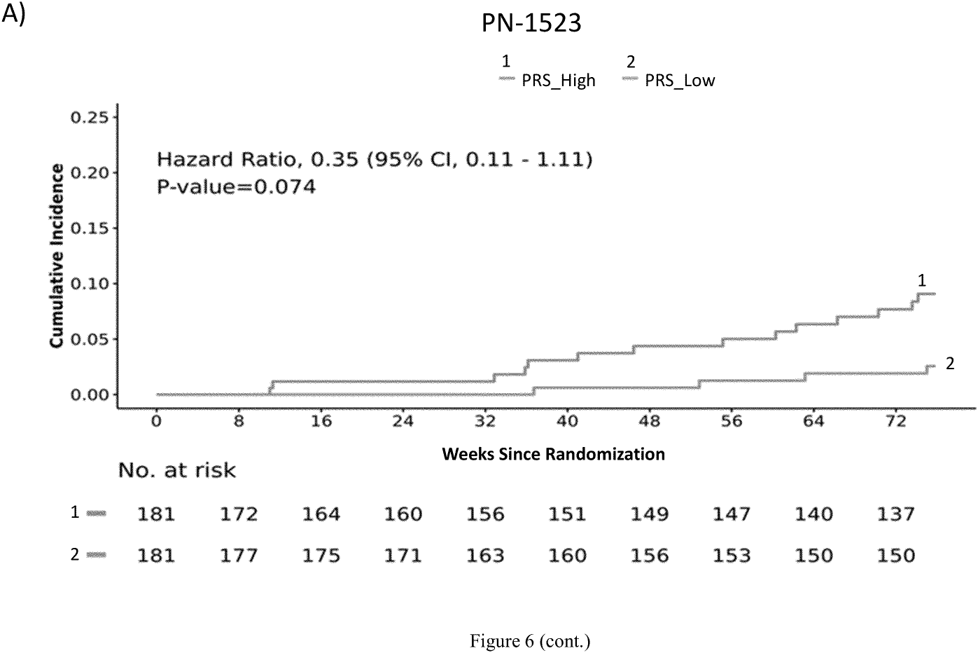

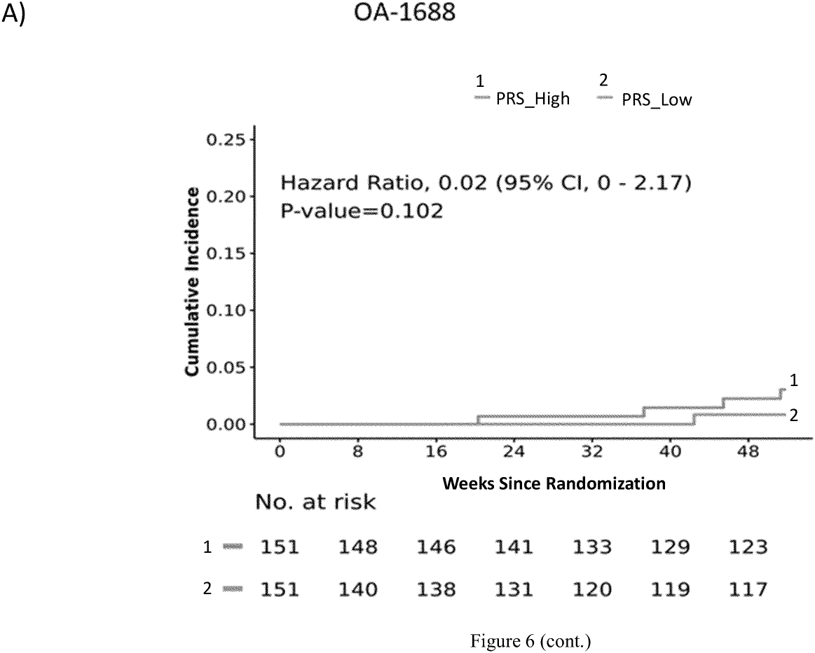

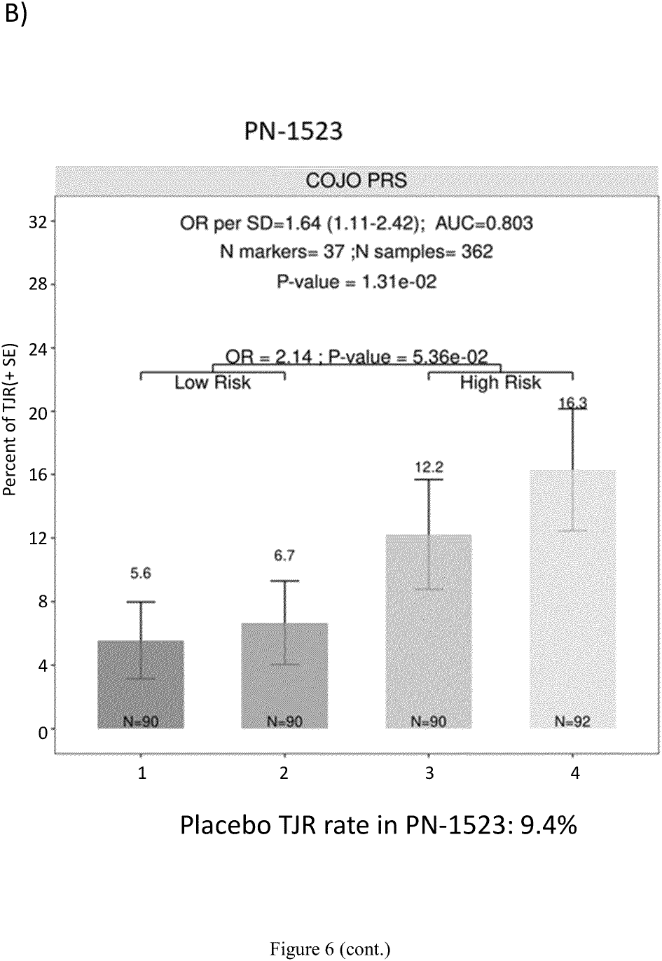

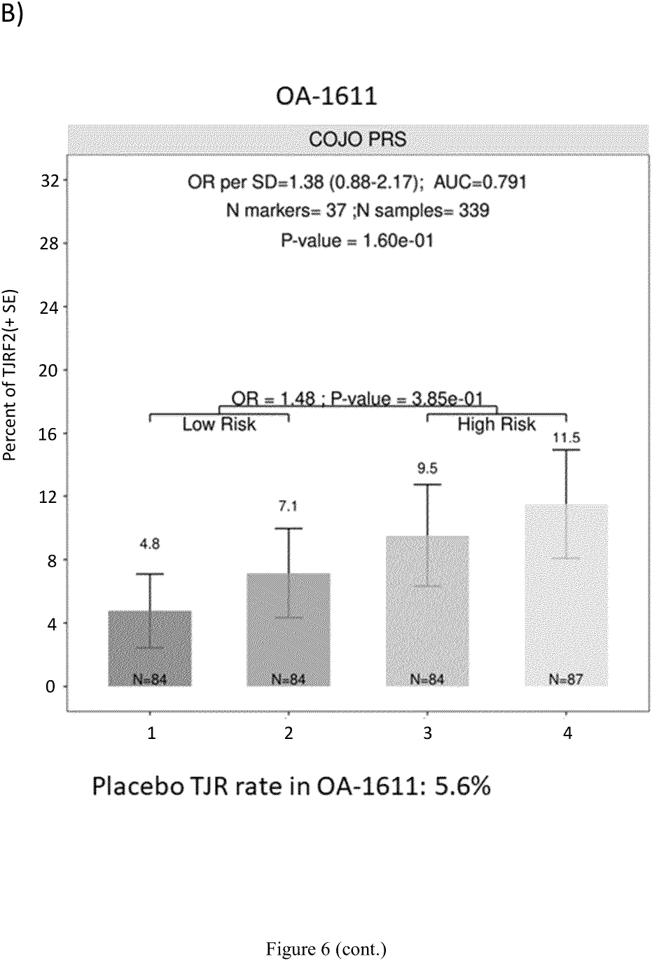

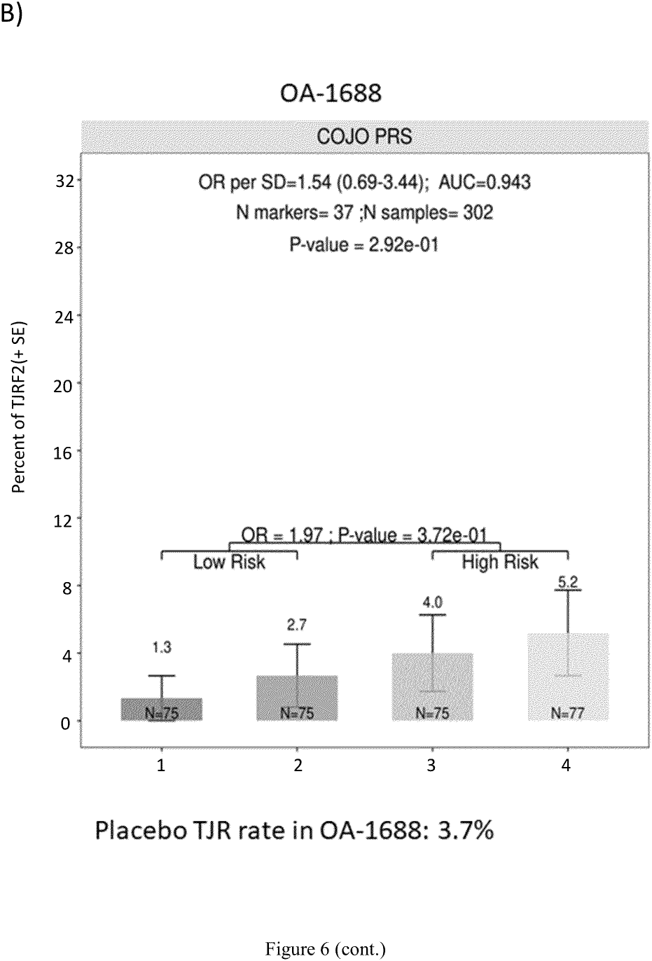

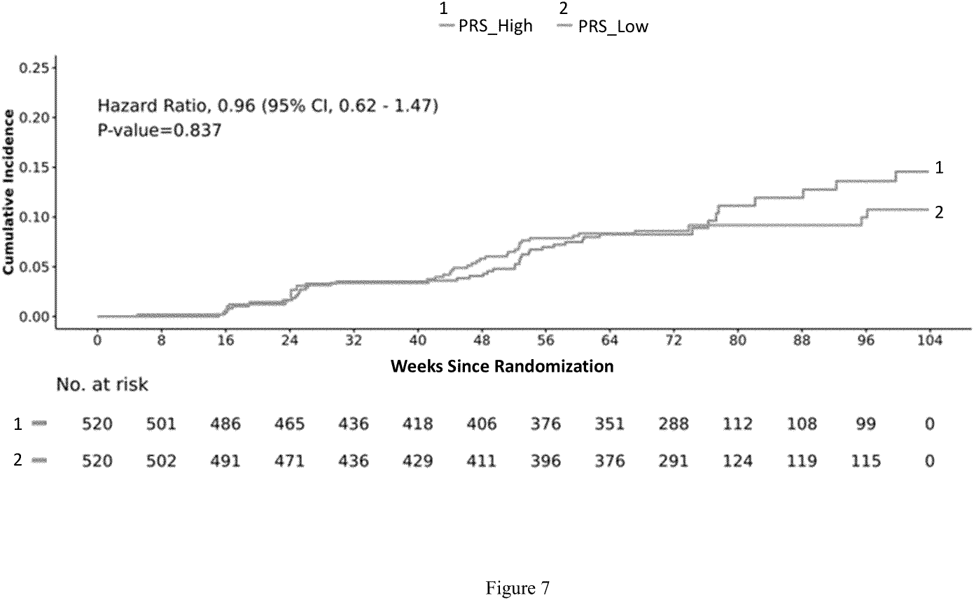

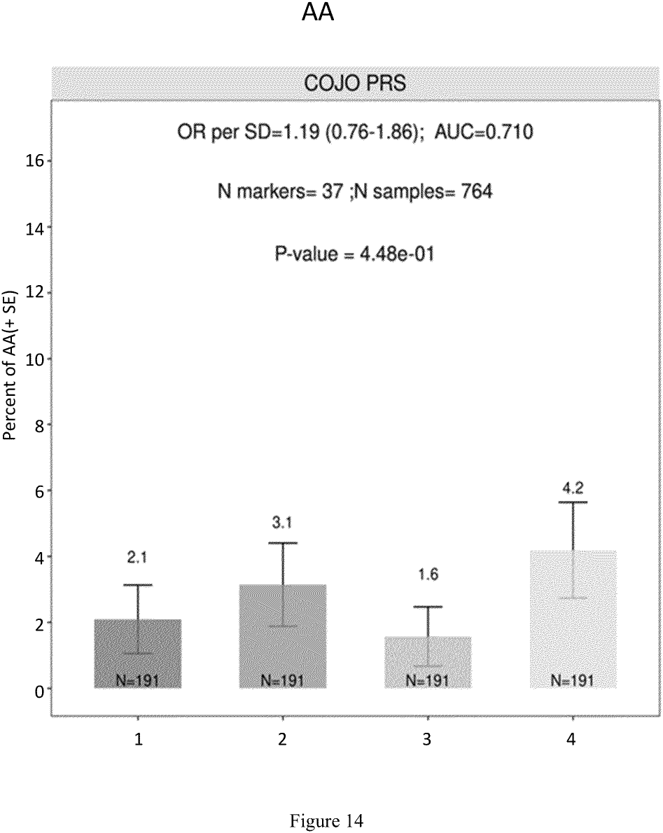

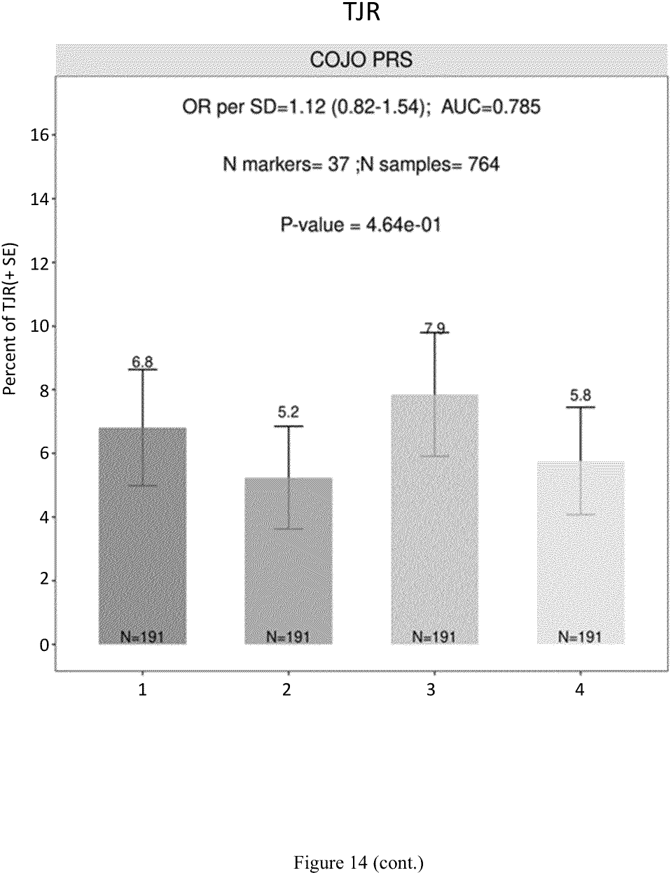

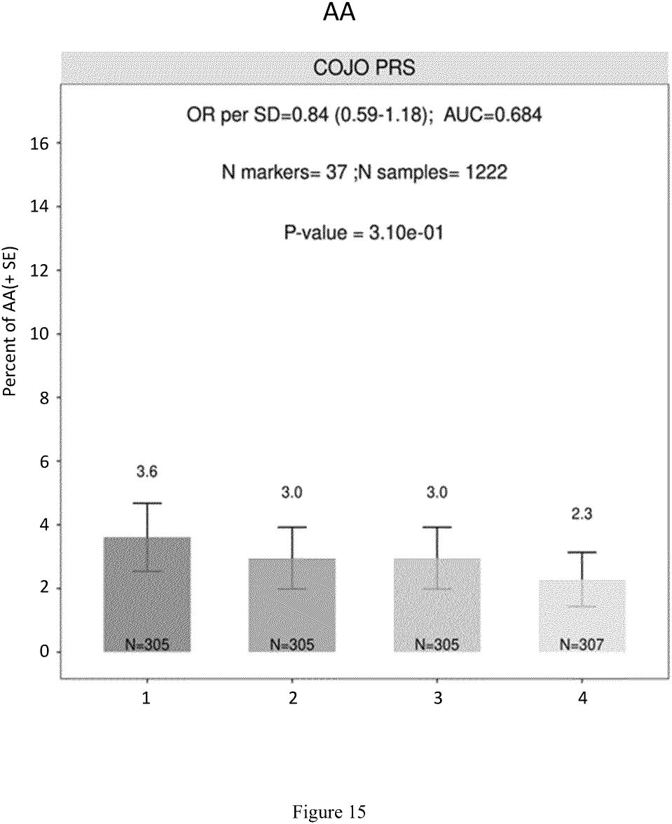

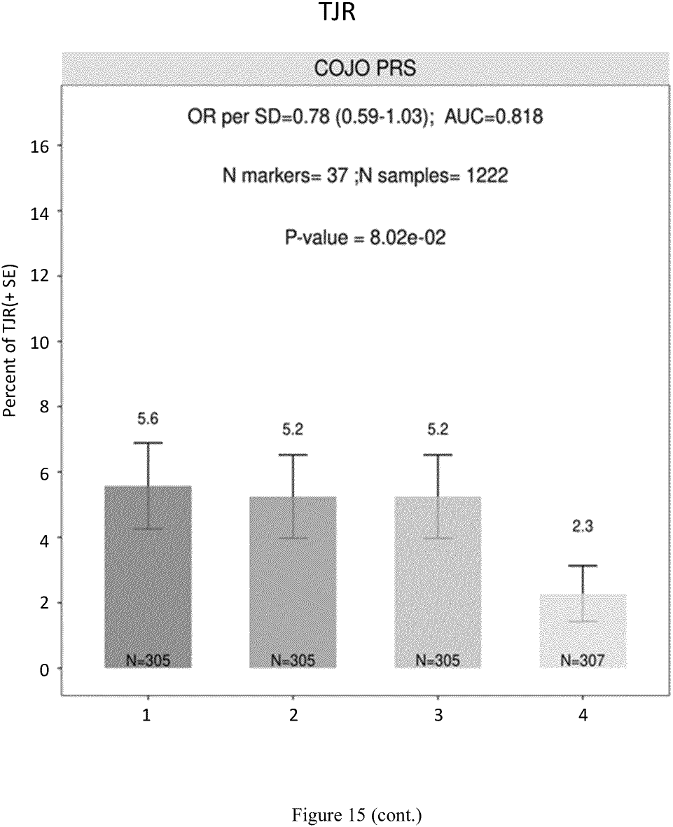

Patients treated with fasinumab 1 mg Q4W with high PRS scores had an increased risk for developing AA (see, FIG. 4) and TJR (see, FIG. 4) events. OA PRS with cumulative incidence of TJR events in fasinumab 1 mg Q4W treated patients was also examined (see, FIG. 5) showing that patients with high OA PRS risk (above the median OA-PRS) have and increase in TJR events. FIG. 6 shows knee/hip OA PRS with cumulative incidence of TJR events by study (OA-1611, OA-1688, and PN1523) in fasinumab 1 mg Q4W treated patients having 1-2 OA joints (Panel A) and knee/hip OA-PRS by quartiles versus TJR events by study (OA-1611, OA-1688, and PN1523) in fasinumab 1 mg Q4W treated patients having 1-2 OA joints (Panel B). In each clinical trial, patients with high OA PRS scores have increased risk for TJR events. Similar results were obtained in fasinumab 1 mg Q4W treated patients for AA (see FIG. 7). FIG. 7 shows knee/hip OA PRS cumulative incidence of AA events in fasinumab 1 mg Q4W treated patients having 1-2 OA joints. In contrast, little association was observed in placebo treated patients for AA and for TJR (see, FIG. 14). No significant association between OA-PRS was observed in NSAID treated patients for AA but there was an association seen for TJR (see, FIG. 15) with high OA-PRS patients having a decreased risk for TJR.

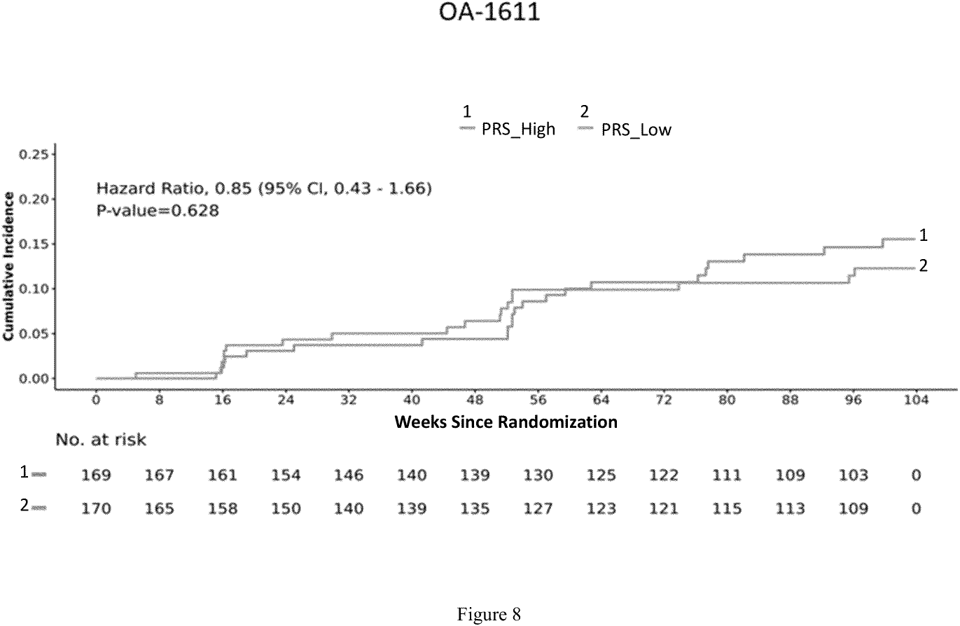

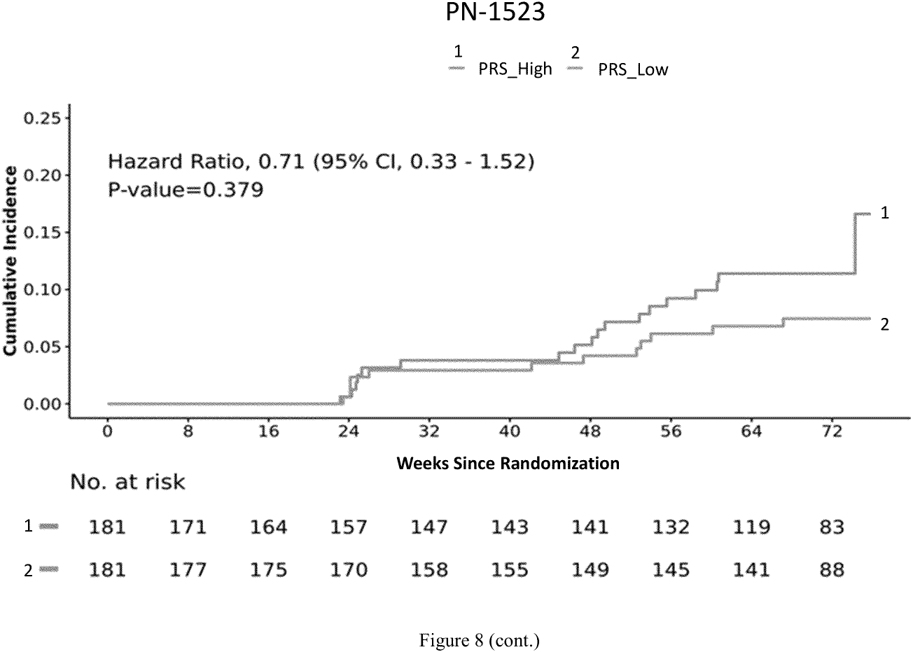

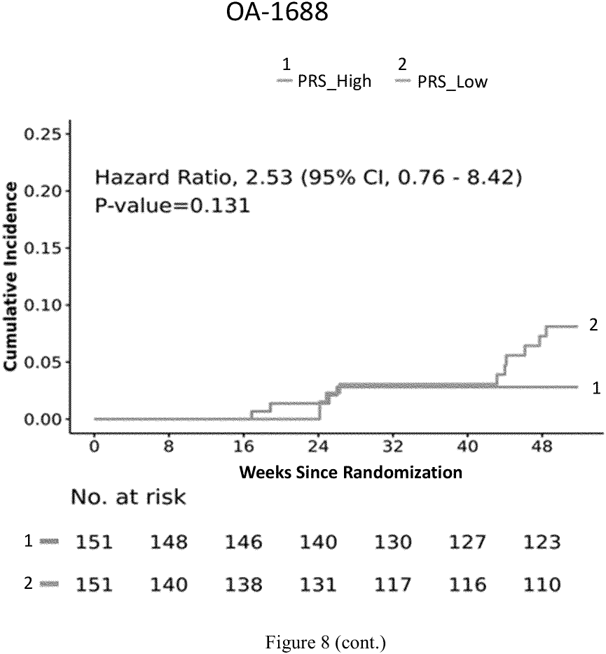

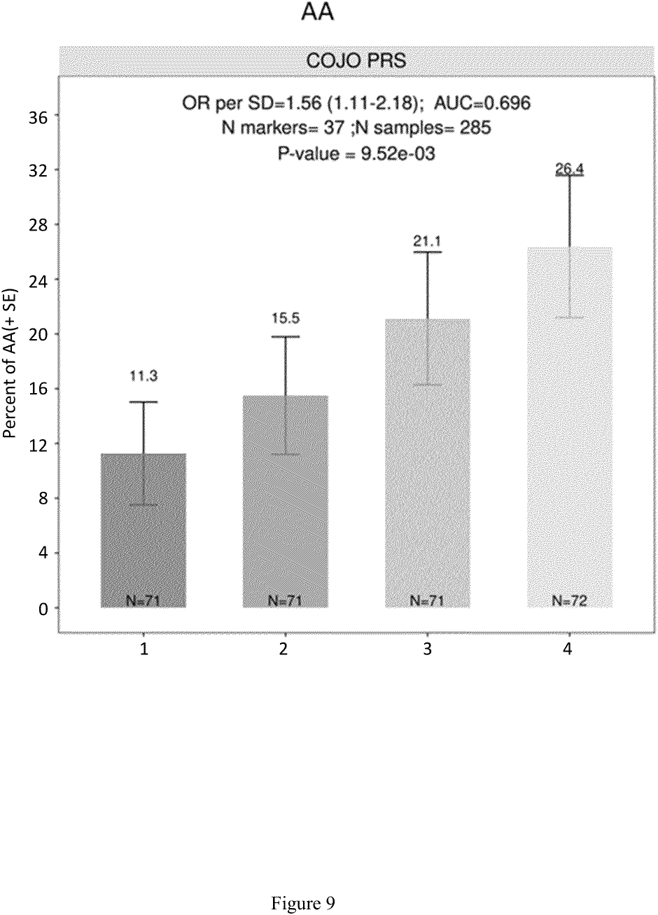

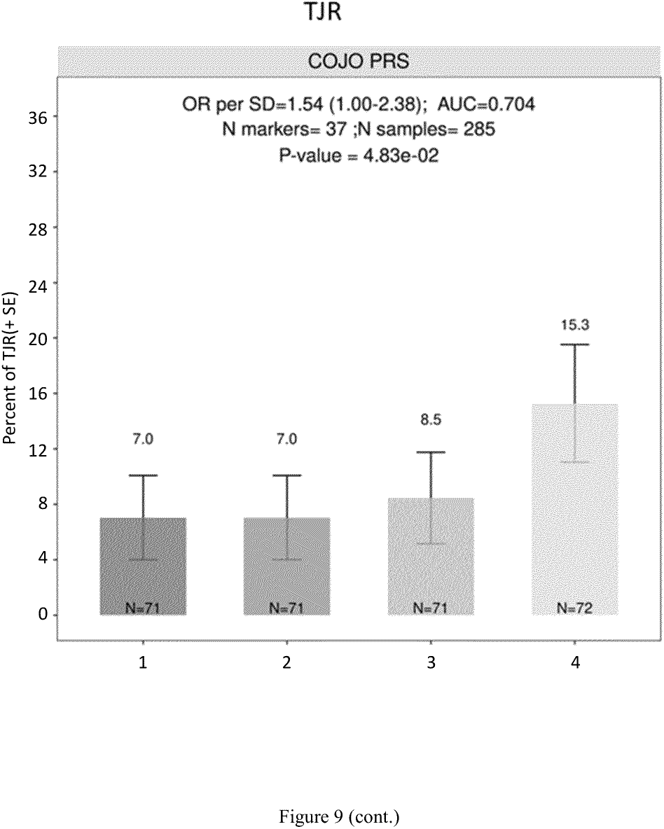

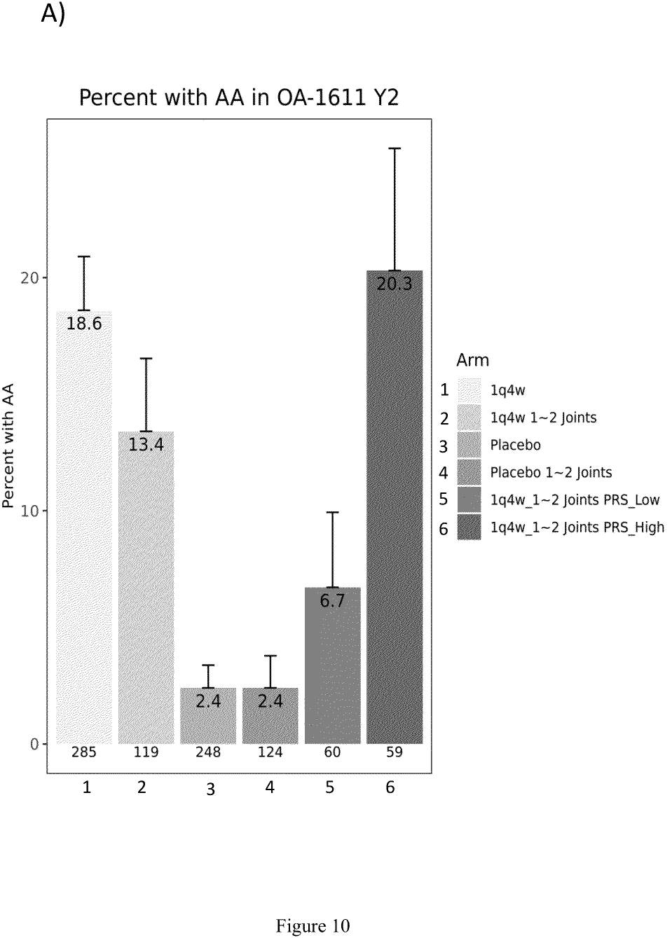

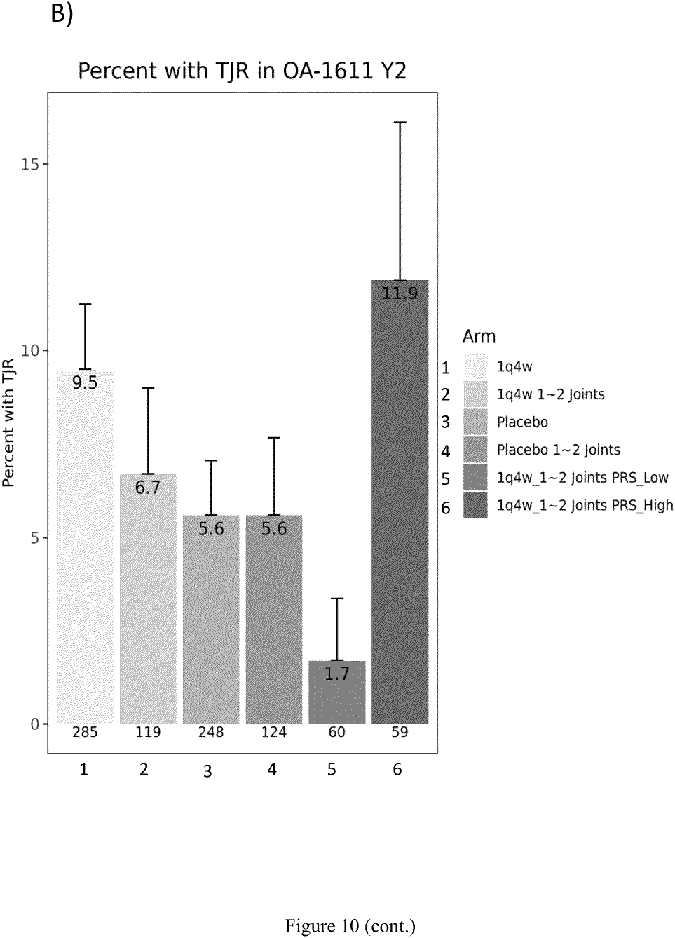

The increase in AA risk in patients with high OA-PRS scores is seen most strongly after treatment for longer durations. FIG. 8 shows knee/hip OA PRS cumulative incidence of AA in fasinumab 1 mg Q4W treated patients having 1-2 OA joints by individual study. Trials OA-1611 and PN-1523 with longer durations of treatment show an increased risk for AA events for high PRS patients while study 1688, with shorter treatment duration, does not show an association for AA events. Study 1611 years 2 treatment period (FIG. 9) shows a strong association for knee/hip OA PRS and both AA and TJR events in patients under fasinumab 1 mg Q4W treatment (1611 Year 2 study). A comparison between rates in 1 mg Q4W, 1 mg Q4W 1-2 joints, and placebo, low risk OA-PRS individuals show a large decrease in AA events (6.7%) compared to high OA-PRS risk patients (FIG. 10, Panel A) in 1611 trial year 2 data. A comparison between rates in 1 mg Q4W, 1 mg Q4W 1-2 joints, and placebo, low risk OA-PRS individuals show a very large decrease in TJR events (1.7%) compared to high OA-PRS risk patients (FIG. 10, Panel B) in 1611 trial year 2 data. These rates are considerably smaller to what is observed in the placebo treated patients (5.6%) suggesting the OA-PRS score may identify a set of patients that greatly benefit from NGF treatment and have lower TJR events.

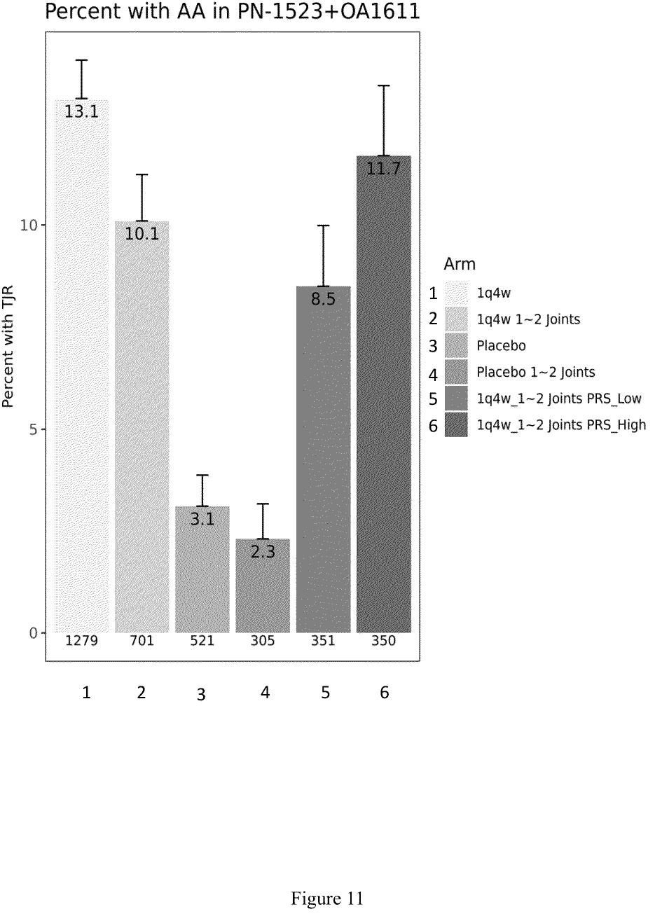

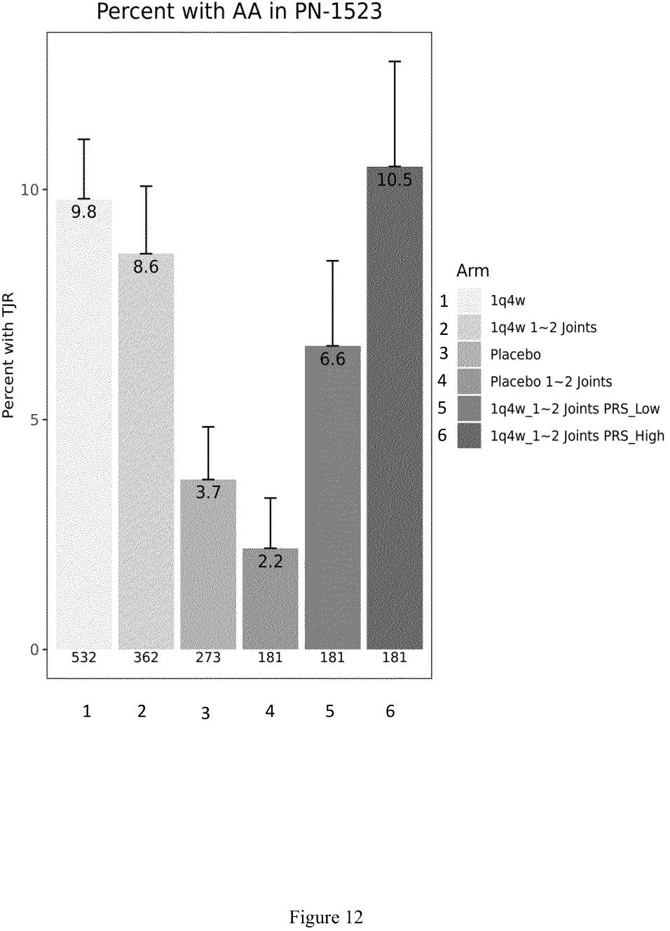

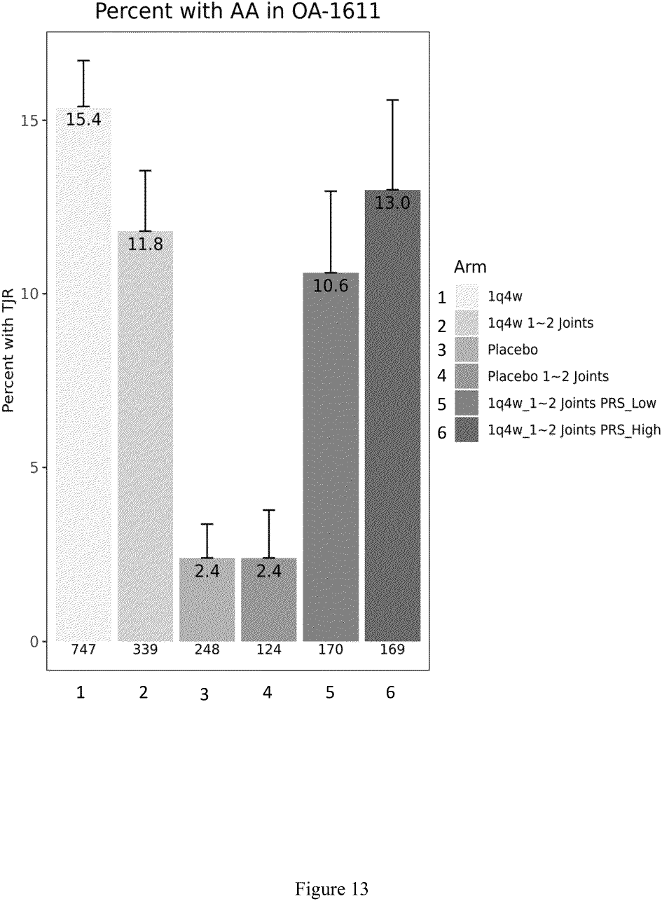

Combining long term trials of OA-1611 and PN-1523 shows a decrease in AA event in low OA-PRS risk patients (FIG. 11) with low dose (1 mg Q4W) and lower OA joint counts (1-2 joints). Similar OA-PRS results are seen for PN-1523 study alone (FIG. 12) and OA-1611 (FIG. 13) with low dose (1 mg Q4W) and lower OA joint counts (1-2 joints). In summary, patients with high OA PRS scores are at an increased risk for developing AA and TJR after fasinumab treatment, and limiting treatment of fasinumab to patients with lower OA-PRS scores would improve patient safety.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method of treating a subject having osteoarthritis (OA), or at risk of developing OA, the method comprising:
administering an analgesic and a therapeutic agent that treats OA to the subject when the subject’s osteoarthritis polygenic risk score (OA-PRS) is greater than or equal to a threshold OA-PRS; or
administering a Nerve Growth Factor (NGF) antagonist and a therapeutic agent that treats OA to the subject when the subject’s OA-PRS is less than a threshold OA-PRS;
wherein the OA-PRS comprises a weighted aggregate of a plurality of genetic variants associated with Total Joint Replacement (TJR) and/or Adjudicated Arthropathy (AA) in subjects treated with an NGF antagonist.

2. A method of treating a subject having osteoarthritis (OA), or at risk of developing OA, the method comprising:
administering an analgesic in a standard dosage amount or greater and administering a therapeutic agent that treats OA to the subject when the subject’s osteoarthritis polygenic risk score (OA-PRS) is greater than or equal to a threshold OA-PRS,
wherein the OA-PRS comprises a weighted aggregate of a plurality of genetic variants associated with Total Joint Replacement (TJR) and/or Adjudicated Arthropathy (AA) in subjects treated with an NGF antagonist.

3-5. (canceled)

6. The method according to claim 1, wherein the NGF antagonist is an antibody, a polypeptide, an antisense nucleic acid molecule, an siRNA molecule, or a small molecule.

7. The method according to claim 6, wherein the antibody is fasinumab, tanezumab, or fulranumab.

8. The method according to claim 7, wherein the antibody is fasinumab.

9. The method according to claim 6, wherein the small molecule is K252a, ALE-0540, PQC-083, PD-90780, LM11A-31 dihydrochloride, Y1036, GZ389988A, or Ro 08-2750.

10. The method according to claim 1, wherein the threshold OA-PRS is the top 75% or the top quintile within a reference population.

11. (canceled)

12. The method according to claim 1, wherein an endpoint of the OA-PRS is a total joint replacement or adjudicated arthropathy.

13. (canceled)

14. The method according to claim 1, wherein the reference population comprises at least 100 subjects, at least 1,000 subjects, at least 5,000 subjects, or at least 10,000 subjects.

15-17. (canceled)

18. The method according to claim 1, wherein the reference population is enriched for members of an ancestry group.

19. The method according to claim 18, wherein the ancestry group comprises a European ancestry group, an African ancestry group, an admixed American ancestry group, an East Asian ancestry group, or a South Asian ancestry group.

20. The method according to claim 18, wherein a member of the ancestry group has self-reported membership of the ancestry group or is determined by genetic testing for ancestry.

21. (canceled)

22. The method according to claim 1, wherein the plurality of genetic variants comprises a single nucleotide polymorphism (SNP), an insertion, a deletion, a structural variant, or a copy-number variation.

23. The method according to claim 1, wherein the plurality of genetic variants is determined by calculating a genetic variant performance in the reference population and selecting the highest performing genetic variants.

24. The method according to claim 23, wherein the genetic variant performance is calculated with respect to a strength of association and/or a probability distribution.

25. The method according to claim 1, wherein the OA-PRS is calculated using an LDPred method, Pruning and Thresholding method, COJO method, or SBayesR method.

26. The method according to claim 1, wherein the plurality of genetic variants comprises at least 2 genetic variants, at least 4 genetic variants, at least 150 genetic variants, at least 500 genetic variants, at least 1,000 genetic variants, at least 10,000 genetic variants, at least 100,000 genetic variants, at least 1,000,000 genetic variants, or at least 10,000,000 genetic variants.

27-34. (canceled)

35. The method according to claim 1, wherein the OA-PRS is determined from a biological sample obtained from the subject, wherein the biological sample comprises blood, semen, saliva, urine, feces, hair, teeth, bone, tissue, a swab from a cheek, or a cell.

36. The method according to claim 35, wherein the biological sample comprises blood.

37. The method according to claim 1, wherein the subject has had administered or is currently being administered fasinumab.
